(19) United States
(12) Patent Application Publication
(10) Pub. No.: US 2005/0123971 A1
(43) Pub. Date: Jun. 9, 2005

(54) System, method, and computer software product for generating genotype calls

(75) Inventors: Xiaojun Di, Sunnyvale, CA (US); Teresa A. Webster, Olga, WA (US); Daniel M. Bartell, San Carlos, CA (US); Richard Chiles, Castro Valley, CA (US)

Correspondence Address: AFFYMETRIX, INC;ATTN: CHIEF IP COUNSEL, LEGAL DEPT., 3380 CENTRAL EXPRESSWAY, SANTA CLARA, CA, 95051, US

(73) Assignee: Affymetrix, INC., Santa Clara, CA (US)

(21) Appl. No.: 10/986,963

(22) Filed: Nov. 12, 2004

Related U.S. Application Data
(63) Continuation-in-part of application No. 10/657,481, filed on Sep. 8, 2003.
(60) Provisional application No. 60/519,146, filed on Nov. 12, 2003.
(60) Provisional application No. 60/519,570, filed on Nov. 12, 2003.
(60) Provisional application No. 60/578,816, filed on Jun. 10, 2004.
(60) Provisional application No. 60/581,773, filed on Jun. 22, 2004.

(51) Int. Cl. C12Q 1/68; G06F 19/00; G01N 33/48; G01N 33/50

(52) U.S. Cl. 435/6; 702/20

(57) ABSTRACT

A method for calling the genotype of a sample is described comprising the acts of receiving emission data for one or more target sequences each hybridized to a plurality of probe sets, where each of the probe sets comprises a plurality of probe features; calculating a set of values for each of the probe sets associated with each target sequence; selecting one of the set of values for each of the probe sets associated with each target sequence, wherein the value is selected if it is greater than a reference value; determining a significance value from the selected values of all the probe sets associated with each target sequence; and producing a genotype call for each target sequence based upon the significance value.

### RELATED APPLICATIONS

The present application claims priority to and is a Continuation-In-Part of U.S. patent application Ser. No. 10/657,481, titled “System, Method, and Computer Software Product for Analysis and Display of Genotyping, Annotation, and Related Information”, filed Sep. 8, 2003; U.S. Provisional Patent Application Ser. No. 60/519,146, titled “System, Method, and Computer Software Product for A Dynamic Model Based Genotyping Algorithm and Genotype Data Visualization for the Determination and Comparison of Biological Sequence Composition”, filed Nov. 12, 2003; U.S. Provisional Patent Application Ser. No. 60/519,570, titled “System, Method, and Computer Software Product for A Dynamic Model Based Genotyping Algorithm and Genotype Data Visualization for the Determination and Comparison of Biological Sequence Composition”, filed Nov. 12, 2003; U.S. Provisional Patent Application Ser. No. 60/578,816, titled “System, Method, and Computer Software Product for Genotyping and Genotype Data Visualization”, filed Jun. 10, 2004; and U.S. Provisional Patent Application Ser. No. 60/581,773, titled “System and Method for Improved Genotype Calls Using Microarrays”, filed Jun. 22, 2004; each of which is hereby incorporated by reference herein in its entirety for all purposes.

### BACKGROUND

1. Field of the Invention

The present invention relates to the field of bioinformatics. In particular, the present invention relates to computer systems, methods, and products for the storage and presentation of data resulting from the analysis of microarrays of biological materials.

2. Related Art

Synthesized nucleic acid probe arrays, such as Affymetrix® GeneChip® probe arrays, and spotted probe arrays, have been used to generate unprecedented amounts of information about biological systems. For example, the GeneChip® Human Genome U133 Plus 2.0 probe array available from Affymetrix, Inc. of Santa Clara, Calif., is comprised of a single microarray containing over 1,000,000 unique oligonucleotide features covering more than 47,000 transcripts that represent more than 33,000 human genes. Analysis of expression data from such microarrays may lead to the development of new drugs and new diagnostic tools.

### SUMMARY OF THE INVENTION

Systems, methods, and products to address these and other needs are described herein with respect to illustrative, non-limiting, implementations. Various alternatives, modifications and equivalents are possible. For example, certain systems, methods, and computer software products are described herein using exemplary implementations for analyzing data from arrays of biological materials produced by the Affymetrix® 417™ or 427™ Arrayer. Other illustrative implementations are referred to in relation to data from Affymetrix® GeneChip® probe arrays. However, these systems, methods, and products may be applied with respect to many other types of probe arrays and, more generally, with respect to numerous parallel biological assays produced in accordance with other conventional technologies and/or produced in accordance with techniques that may be developed in the future. For example, the systems, methods, and products described herein may be applied to parallel assays of nucleic acids, PCR products generated from cDNA clones, proteins, antibodies, or many other biological materials. These materials may be disposed on slides (as typically used for spotted arrays), on substrates employed for GeneChip® arrays, or on beads, optical fibers, or other substrates or media, which may include polymeric coatings or other layers on top of slides or other substrates. Moreover, the probes need not be immobilized in or on a substrate, and, if immobilized, need not be disposed in regular patterns or arrays. For convenience, the term “probe array” will generally be used broadly hereafter to refer to all of these types of arrays and parallel biological assays.

A method for calling the genotype of a sample is described comprising the acts of receiving emission data for one or more target sequences each hybridized to a plurality of probe sets, where each of the probe sets comprises a plurality of probe features; calculating a set of values for each of the probe sets associated with each target sequence; selecting one of the set of values for each of the probe sets associated with each target sequence, wherein the value is selected if it is greater than a reference value; determining a significance value from the selected values of all the probe sets associated with each target sequence; and producing a genotype call for each target sequence based upon the significance value.

In some implementations, each of the set of values is calculated based upon one or more assumptions, such as an assumption of a genotype that may include a null assumption, a homozygous assumption, and a heterozygous assumption.

Also, a computer for calling the genotype of a sample is described comprising system memory with executable code stored thereon, where the executable code is enabled to perform a method, comprising the acts of receiving emission data for one or more target sequences each hybridized to a plurality of probe sets, where each of the probe sets comprises a plurality of probe features; calculating a set of values for each of the probe sets associated with each target sequence; selecting one of the set of values for each of the probe sets associated with each target sequence, wherein the value is selected if it is greater than a reference value; determining a significance value from the selected values of all the probe sets associated with each target sequence; and producing a genotype call for each target sequence based upon the significance value.

The above implementations are not necessarily inclusive or exclusive of each other and may be combined in any manner that is non-conflicting and otherwise possible, whether they are presented in association with a same, or a different, aspect of implementation. The description of one implementation is not intended to be limiting with respect to other implementations. Also, any one or more function, step, operation, or technique described elsewhere in this specification may, in alternative implementations, be combined with any one or more function, step, operation, or technique described in the summary. Thus, the above implementations are illustrative rather than limiting.

### BRIEF DESCRIPTION OF THE DRAWINGS

In the drawings, like reference numerals indicate like structures or method steps and the leftmost digit of a reference numeral indicates the number of the figure in which the referenced element first appears (for example, the element 120 appears first in FIG. 1). In functional block diagrams, rectangles generally indicate functional elements, parallelograms generally indicate data, and rectangles with a pair of double borders generally indicate predefined functional elements. These conventions, however, are intended to be typical or illustrative, rather than limiting.

FIG. 1 is a functional block diagram of one embodiment of a computer system including illustrative embodiments of probe array analysis executables and display/output devices including graphical user interfaces;

FIG. 2 is a functional block diagram of one embodiment of the computer system of FIG. 1 connected to a user-side Internet client and database server via a network for communication over the Internet;

FIG. 3 is a functional block diagram of one embodiment of the probe array analysis executables of FIG. 1 including illustrative embodiments of a sequence data manager and an output manager;

FIG. 4 is a simplified graphical representation of one embodiment of a GUI which displays a plurality of genotype calls associated with multiple samples in a two dimensional format;

FIG. 5 is a simplified graphical representation of one embodiment of an interactive GUI depicting a map that graphically displays the plurality of genotype calls associated with multiple samples;

FIG. 6 is a simplified graphical representation of one embodiment of an interactive GUI displaying the map of FIG. 5 where the display is based, at least in part, upon a change of one or more parameters;

FIG. 7 is a simplified graphical representation of one embodiment of a GUI that displays the maps of FIGS. 5, and 6 that provides one or more graphical elements associated with the genotype calls; and

FIG. 8 is a simplified graphical representation of one embodiment of a GUI that displays the maps of FIGS. 5, 6, and 7, 8 that provides a graphical illustration based, at least in part, upon statistical analysis of the data;

### DETAILED DESCRIPTION

### a) General

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3^{rd} Ed., W.H. Freeman Pub., New York, N.Y. and Berg et al. (2002) Biochemistry, 5^{th} Ed., W.H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication Number WO 99/36760) and PCT/US01/04285 (International Publication Number WO 01/58593), which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

Nucleic acid arrays that are useful in the present invention include those that are commercially available from Affymetrix (Santa Clara, Calif.) under the brand name GeneChip®. Example arrays are shown on the website at affymetrix.com.

The present invention also contemplates many uses for polymers attached to solid substrates. These uses include gene expression monitoring, profiling, library screening, genotyping and diagnostics. Gene expression monitoring and profiling methods can be shown in U.S. Pat. Nos. 5,800,992, 6,013,449, 6,020,135, 6,033,860, 6,040,138, 6,177,248 and 6,309,822. Genotyping and uses therefore are shown in U.S. Ser. Nos. 10/442,021, 10/013,598 (U.S. Patent Application Publication 20030036069), and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with genotyping, the genomic sample may be amplified by a variety of mechanisms, some of which may employ PCR. See, e.g., PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675, and each of which is incorporated herein by reference in their entireties for all purposes. The sample may be amplified on the array. See, for example, U.S. Pat. No. 6,300,070 and U.S. Ser. No. 09/513,300, which are incorporated herein by reference.

Other suitable amplification methods include the ligase chain reaction (LCR) (e.g., Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. Nos. 6,361,947, 6,391,592 and U.S. Ser. Nos. 09/916,135, 09/920,491 (U.S. Patent Application Publication 20030096235), Ser. No. 09/910,292 (U.S. Patent Application Publication 20030082543), and Ser. No. 10/013,598.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2^{nd} Ed. Cold Spring Harbor, N.Y., 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference

The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. Ser. No. 10/389,194 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Ser. Nos. 10/389,194, 60/493,495 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001). See U.S. Pat. No. 6,420,108.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over networks such as the Internet as shown in U.S. Ser. Nos. 10/197,621, 10/063,559 (United States Publication No. 20020183936), Ser. Nos. 10/065,856, 10/065,868, 10/328,818, 10/328,872, 10/423,403, and 60/482,389.

### b) Definitions

An “array” is an intentionally created collection of molecules which can be prepared either synthetically or biosynthetically. The molecules in the array can be identical or different from each other. The array can assume a variety of formats, e.g., libraries of soluble molecules; libraries of compounds tethered to resin beads, silica chips, or other solid supports.

Nucleic acid library or array is an intentionally created collection of nucleic acids which can be prepared either synthetically or biosynthetically and screened for biological activity in a variety of different formats (e.g., libraries of soluble molecules; and libraries of oligos tethered to resin beads, silica chips, or other solid supports). Additionally, the term “array” is meant to include those libraries of nucleic acids which can be prepared by spotting nucleic acids of essentially any length (e.g., from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

Biopolymer or biological polymer: is intended to mean repeating units of biological or chemical moieties. Representative biopolymers include, but are not limited to, nucleic acids, oligonucleotides, amino acids, proteins, peptides, hormones, oligosaccharides, lipids, glycolipids, lipopolysaccharides, phospholipids, synthetic analogues of the foregoing, including, but not limited to, inverted nucleotides, peptide nucleic acids, Meta-DNA, and combinations of the above. “Biopolymer synthesis” is intended to encompass the synthetic production, both organic and inorganic, of a biopolymer.

Related to a bioploymer is a “biomonomer” which is intended to mean a single unit of biopolymer, or a single unit which is not part of a biopolymer. Thus, for example, a nucleotide is a biomonomer within an oligonucleotide biopolymer, and an amino acid is a biomonomer within a protein or peptide biopolymer; avidin, biotin, antibodies, antibody fragments, etc., for example, are also biomonomers. initiation Biomonomer: or “initiator biomonomer” is meant to indicate the first biomonomer which is covalently attached via reactive nucleophiles to the surface of the polymer, or the first biomonomer which is attached to a linker or spacer arm attached to the polymer, the linker or spacer arm being attached to the polymer via reactive nucleophiles.

Complementary: Refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

Combinatorial Synthesis Strategy: A combinatorial synthesis strategy is an ordered strategy for parallel synthesis of diverse polymer sequences by sequential addition of reagents which may be represented by a reactant matrix and a switch matrix, the product of which is a product matrix. A reactant matrix is a 1 column by m row matrix of the building blocks to be added. The switch matrix is all or a subset of the binary numbers, preferably ordered, between 1 and m arranged in columns. A “binary strategy” is one in which at least two successive steps illuminate a portion, often half, of a region of interest on the substrate. In a binary synthesis strategy, all possible compounds which can be formed from an ordered set of reactants are formed. In most preferred embodiments, binary synthesis refers to a synthesis strategy which also factors a previous addition step. For example, a strategy in which a switch matrix for a masking strategy halves regions that were previously illuminated, illuminating about half of the previously illuminated region and protecting the remaining half (while also protecting about half of previously protected regions and illuminating about half of previously protected regions). It will be recognized that binary rounds may be interspersed with non-binary rounds and that only a portion of a substrate may be subjected to a binary scheme. A combinatorial “masking” strategy is a synthesis which uses light or other spatially selective deprotecting or activating agents to remove protecting groups from materials for addition of other materials such as amino acids.

Effective amount refers to an amount sufficient to induce a desired result.

Genome is all the genetic material in the chromosomes of an organism. DNA derived from the genetic material in the chromosomes of a particular organism is genomic DNA. A genomic library is a collection of clones made from a set of randomly generated overlapping DNA fragments representing the entire genome of an organism.

Hybridization conditions will typically include salt concentrations of less than about 1M, more usually less than about 500 mM and preferably less than about 200 mM. Hybridization temperatures can be as low as 5.degree. C., but are typically greater than 22.degree. C., more typically greater than about 30.degree. C., and preferably in excess of about 37.degree. C. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone.

Hybridizations, e.g., allele-specific probe hybridizations, are generally performed under stringent conditions. For example, conditions where the salt concentration is no more than about 1 Molar (M) and a temperature of at least 25 degrees-Celsius (° C.), e.g., 750 mM NaCl, 50 mM NaPhosphate, 5 mM EDTA, pH 7.4 (5×SSPE) and a temperature of from about 25 to about 30° C.

Hybridizations are usually performed under stringent conditions, for example, at a salt concentration of no more than 1 M and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations. For stringent conditions, see, for example, Sambrook, Fritsche and Maniatis. “Molecular Cloning A laboratory Manual” 2^{nd} Ed. Cold Spring Harbor Press (1989) which is hereby incorporated by reference in its entirety for all purposes above.

The term “hybridization” refers to the process in which two single-stranded polynucleotides bind non-covalently to form a stable double-stranded polynucleotide; triple-stranded hybridization is also theoretically possible. The resulting (usually) double-stranded polynucleotide is a “hybrid.” The proportion of the population of polynucleotides that forms stable hybrids is referred to herein as the “degree of hybridization.”

Hybridization probes are oligonucleotides capable of binding in a base-specific manner to a complementary strand of nucleic acid. Such probes include peptide nucleic acids, as described in Nielsen et al., Science 254, 1497-1500 (1991), and other nucleic acid analogs and nucleic acid mimetics.

Hybridizing specifically to: refers to the binding, duplexing, or hybridizing of a molecule only to a particular nucleotide sequence or sequences under stringent conditions when that sequence is present in a complex mixture (e.g., total cellular) DNA or RNA.

Isolated nucleic acid is an object species invention that is the predominant species present (i.e., on a molar basis it is more abundant than any other individual species in the composition). Preferably, an isolated nucleic acid comprises at least about 50, 80 or 90% (on a molar basis) of all macromolecular species present. Most preferably, the object species is purified to essential homogeneity (contaminant species cannot be detected in the composition by conventional detection methods).

Ligand: A ligand is a molecule that is recognized by a particular receptor. The agent bound by or reacting with a receptor is called a “ligand,” a term which is definitionally meaningful only in terms of its counterpart receptor. The term “ligand” does not imply any particular molecular size or other structural or compositional feature other than that the substance in question is capable of binding or otherwise interacting with the receptor. Also, a ligand may serve either as the natural ligand to which the receptor binds, or as a functional analogue that may act as an agonist or antagonist. Examples of ligands that can be investigated by this invention include, but are not restricted to, agonists and antagonists for cell membrane receptors, toxins and venoms, viral epitopes, hormones (e.g., opiates, steroids, etc.), hormone receptors, peptides, enzymes, enzyme substrates, substrate analogs, transition state analogs, cofactors, drugs, proteins, and antibodies.

Linkage disequilibrium or allelic association means the preferential association of a particular allele or genetic marker with a specific allele, or genetic marker at a nearby chromosomal location more frequently than expected by chance for any particular allele frequency in the population. For example, if locus X has alleles a and b, which occur equally frequently, and linked locus Y has alleles c and d, which occur equally frequently, one would expect the combination ac to occur with a frequency of 0.25. If ac occurs more frequently, then alleles a and c are in linkage disequilibrium. Linkage disequilibrium may result from natural selection of certain combination of alleles or because an allele has been introduced into a population too recently to have reached equilibrium with linked alleles.

Mixed population or complex population: refers to any sample containing both desired and undesired nucleic acids. As a non-limiting example, a complex population of nucleic acids may be total genomic DNA, total genomic RNA or a combination thereof. Moreover, a complex population of nucleic acids may have been enriched for a given population but include other undesirable populations. For example, a complex population of nucleic acids may be a sample which has been enriched for desired messenger RNA (mRNA) sequences but still includes some undesired ribosomal RNA sequences (rRNA).

Monomer: refers to any member of the set of molecules that can be joined together to form an oligomer or polymer. The set of monomers useful in the present invention includes, but is not restricted to, for the example of (poly)peptide synthesis, the set of L-amino acids, D-amino acids, or synthetic amino acids. As used herein, “monomer” refers to any member of a basis set for synthesis of an oligomer. For example, dimers of L-amino acids form a basis set of 400 “monomers” for synthesis of polypeptides. Different basis sets of monomers may be used at successive steps in the synthesis of a polymer. The term “monomer” also refers to a chemical subunit that can be combined with a different chemical subunit to form a compound larger than either subunit alone.

mRNA or mRNA transcripts: as used herein, include, but not limited to pre-mRNA transcript(s), transcript processing intermediates, mature mRNA(s) ready for translation and transcripts of the gene or genes, or nucleic acids derived from the mRNA transcript(s). Transcript processing may include splicing, editing and degradation. As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

Nucleic acid library or array is an intentionally created collection of nucleic acids which can be prepared either synthetically or biosynthetically and screened for biological activity in a variety of different formats (e.g., libraries of soluble molecules; and libraries of oligos tethered to resin beads, silica chips, or other solid supports). Additionally, the term “array” is meant to include those libraries of nucleic acids which can be prepared by spotting nucleic acids of essentially any length (e.g., from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

Nucleic acids according to the present invention may include any polymer or oligomer of pyrimidine and purine bases, preferably cytosine, thymine, and uracil, and adenine and guanine, respectively. See Albert L. Lehninger, Principles of Biochemistry, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

An “oligonucleotide” or “polynucleotide” is a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA) which may be isolated from natural sources, recombinantly produced or artificially synthesized and mimetics thereof. A further example of a polynucleotide of the present invention may be peptide nucleic acid (PNA). The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

Probe: A probe is a surface-immobilized molecule that can be recognized by a particular target. See U.S. Pat. No. 6,582,908 for an example of arrays having all possible combinations of probes with 10, 12, and more bases. Examples of probes that can be investigated by this invention include, but are not restricted to, agonists and antagonists for cell membrane receptors, toxins and venoms, viral epitopes, hormones (e.g., opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies.

Primer is a single-stranded oligonucleotide capable of acting as a point of initiation for template-directed DNA synthesis under suitable conditions e.g., buffer and temperature, in the presence of four different nucleoside triphosphates and an agent for polymerization, such as, for example, DNA or RNA polymerase or reverse transcriptase. The length of the primer, in any given case, depends on, for example, the intended use of the primer, and generally ranges from 15 to 30 nucleotides. Short primer molecules generally require cooler temperatures to form sufficiently stable hybrid complexes with the template. A primer need not reflect the exact sequence of the template but must be sufficiently complementary to hybridize with such template. The primer site is the area of the template to which a primer hybridizes. The primer pair is a set of primers including a 5′ upstream primer that hybridizes with the 5′ end of the sequence to be amplified and a 3′ downstream primer that hybridizes with the complement of the 3′ end of the sequence to be amplified.

Polymorphism refers to the occurrence of two or more genetically determined alternative sequences or alleles in a population. A polymorphic marker or site is the locus at which divergence occurs. Preferred markers have at least two alleles, each occurring at frequency of greater than 1%, and more preferably greater than 10% or 20% of a selected population. A polymorphism may comprise one or more base changes, an insertion, a repeat, or a deletion. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wildtype form. Diploid organisms may be homozygous or heterozygous for allelic forms. A diallelic polymorphism has two forms. A triallelic polymorphism has three forms. Single nucleotide polymorphisms (SNPs) are included in polymorphisms.

Receptor: A molecule that has an affinity for a given ligand. Receptors may be naturally-occurring or manmade molecules. Also, they can be employed in their unaltered state or as aggregates with other species. Receptors may be attached, covalently or noncovalently, to a binding member, either directly or via a specific binding substance. Examples of receptors which can be employed by this invention include, but are not restricted to, antibodies, cell membrane receptors, monoclonal antibodies and antisera reactive with specific antigenic determinants (such as on viruses, cells or other materials), drugs, polynucleotides, nucleic acids, peptides, cofactors, lectins, sugars, polysaccharides, cells, cellular membranes, and organelles. Receptors are sometimes referred to in the art as anti-ligands. As the term receptors is used herein, no difference in meaning is intended. A “Ligand Receptor Pair” is formed when two macromolecules have combined through molecular recognition to form a complex. Other examples of receptors which can be investigated by this invention include but are not restricted to those molecules shown in U.S. Pat. No. 5,143,854, which is hereby incorporated by reference in its entirety.

“Solid support”, “support”, and “substrate” are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. See U.S. Pat. No. 5,744,305 for exemplary substrates.

Target: A molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Examples of targets which can be employed by this invention include, but are not restricted to, antibodies, cell membrane receptors, monoclonal antibodies and antisera reactive with specific antigenic determinants (such as on viruses, cells or other materials), drugs, oligonucleotides, nucleic acids, peptides, cofactors, lectins, sugars, polysaccharides, cells, cellular membranes, and organelles. Targets are sometimes referred to in the art as anti-probes. As the term targets is used herein, no difference in meaning is intended. A “Probe Target Pair” is formed when two macromolecules have combined through molecular recognition to form a complex.

### c) Embodiments of the Invention

User Computer 100: User computer 100 may be a computing device specially designed and configured to support and execute some or all of the functions of probe array analysis applications 199, described below. Computer 100 also may be any of a variety of types of general-purpose computers such as a personal computer, network server, workstation, or other computer platform now or later developed. Computer 100 typically includes known components such as a processor 105, an operating system 110, a graphical user interface (GUI) controller 115, a system memory 120, memory storage devices 125, and input-output controllers 130. It will be understood by those skilled in the relevant art that there are many possible configurations of the components of computer 100 and that some components that may typically be included in computer 100 are not shown, such as cache memory, a data backup unit, and many other devices. Processor 105 may be a commercially available processor such as an Itanium® or Pentium® processor made by Intel Corporation, a SPARC® processor made by Sun Microsystems, an Athalon™ or Opteron™ processor made by AMD corporation, or it may be one of other processors that are or will become available. Processor 105 executes operating system 110, which may be, for example, a Windows®-type operating system (such as Windows NT® 4.0 with SP6a, or Windows XP) from the Microsoft Corporation; a Unix® or Linux-type operating system available from many vendors or what is referred to as an open source; another or a future operating system; or some combination thereof. Operating system 110 interfaces with firmware and hardware in a well-known manner, and facilitates processor 105 in coordinating and executing the functions of various computer programs that may be written in a variety of programming languages. Operating system 110, typically in cooperation with processor 105, coordinates and executes functions of the other components of computer 100. Operating system 110 also provides scheduling, input-output control, file and data management, memory management, and communication control and related services, all in accordance with known techniques.

System memory 120 may be any of a variety of known or future memory storage devices. Examples include any commonly available random access memory (RAM), magnetic medium such as a resident hard disk or tape, an optical medium such as a read and write compact disc, or other memory storage device. Memory storage device 125 may be any of a variety of known or future devices, including a compact disk drive, a tape drive, a removable hard disk drive, or a diskette drive. Such types of memory storage device 125 typically read from, and/or write to, a program storage medium (not shown) such as, respectively, a compact disk, magnetic tape, removable hard disk, or floppy diskette. Any of these program storage media, or others now in use or that may later be developed, may be considered a computer program product. As will be appreciated, these program storage media typically store a computer software program and/or data. Computer software programs, also called computer control logic, typically are stored in system memory 120 and/or the program storage device used in conjunction with memory storage device 125.

In some embodiments, a computer program product is described comprising a computer usable medium having control logic (computer software program, including program code) stored therein. The control logic, when executed by processor 105, causes processor 105 to perform functions described herein. In other embodiments, some functions are implemented primarily in hardware using, for example, a hardware state machine. Implementation of the hardware state machine so as to perform the functions described herein will be apparent to those skilled in the relevant arts.

Input-output controllers 130 could include any of a variety of known devices for accepting and processing information from a user, whether a human or a machine, whether local or remote. Such devices include, for example, modem cards, network interface cards, sound cards, or other types of controllers for any of a variety of known input devices 102. Output controllers of input-output controllers 130 could include controllers for any of a variety of known display devices 180 for presenting information to a user, whether a human or a machine, whether local or remote. If one of display devices 180 provides visual information, this information typically may be logically and/or physically organized as an array of picture elements, sometimes referred to as pixels. Graphical user interface (GUI) controller 115 may comprise any of a variety of known or future software programs for providing graphical input and output interfaces between computer 100 and user 175, and for processing user inputs. In the illustrated embodiment, the functional elements of computer 100 communicate with each other via system bus 104. Some of these communications may be accomplished in alternative embodiments using network or other types of remote communications.

As will be evident to those skilled in the relevant art, applications 199, if implemented in software, may be loaded into system memory 120 and/or memory storage device 125 through one of input devices 102. All or portions of applications 199 may also reside in a read-only memory or similar device of memory storage device 125, such devices not requiring that applications 199 first be loaded through input devices 102. It will be understood by those skilled in the relevant art that applications 199, or portions of it, may be loaded by processor 105 in a known manner into system memory 120, or cache memory (not shown), or both, as advantageous for execution.

Scanner 150: Scanner 150 of this example provides an image of hybridized probe-target pairs by detecting fluorescent, radioactive, or other emissions; by detecting transmitted, reflected, or scattered radiation; by detecting electromagnetic properties or characteristics; or by other techniques. These processes or techniques may generally and collectively be referred to hereafter for convenience simply as involving the detection of “emissions.” Various detection schemes are employed depending on the type of emissions and other factors. A typical scheme employs optical and other elements to provide excitation light and to selectively collect the emissions. Also generally included are various light-detector systems employing photodiodes, charge-coupled devices, photomultiplier tubes, or similar devices to register the collected emissions. For example, a scanning system for use with a fluorescent label is described in U.S. Pat. No. 5,143,854, incorporated by reference above. Illustrative scanners or scanning systems that, in various implementations, may include scanner 150 are described in U.S. Pat. Nos. 5,143,854, 5,578,832, 5,631,734, 5,834,758, 5,936,324, 5,981,956, 6,025,601, 6,141,096, 6,185,030, 6,201,639, 6,218,803, and 6,252,236; in PCT Application PCT/US99/06097 (published as WO99/47964); in U.S. patent applications, Ser. Nos. 10/063,284, 09/683,216, 09/683,217, 09/683,219, 09/681,819, and 09/383,986; and in U.S. Provisional Patent Applications Ser. Nos. 60/364,731, and 60/286,578, each of which is hereby incorporated herein by reference in its entirety for all purposes.

Scanner 150 of this non-limiting example provides data representing the intensities (and possibly other characteristics, such as color) of the detected emissions, as well as the locations on the substrate where the emissions were detected. The data typically are stored in a memory device, such as system memory 120 of user computer 150, in the form of a data file. One type of data file, such as image data 176 that could for example be in the form of a “*.cel” file generated by Microarray Suite software available from Affymetrix, Inc., typically includes intensity and location information corresponding to elemental sub-areas of the scanned substrate. In the illustrated example, data 176 could be received by computer 100 where a *.cel file could be generated or the *.cel file could be generated by scanner 150. The term “elemental” in this context means that the intensities, and/or other characteristics, of the emissions from this area each are represented by a single value. When displayed as an image for viewing or processing, elemental picture elements, or pixels, often represent this information. Thus, for example, a pixel may have a single value representing the intensity of the elemental sub-area of the substrate from which the emissions were scanned. The pixel may also have another value representing another characteristic, such as color. For instance, a scanned elemental sub-area in which high-intensity emissions were detected may be represented by a pixel having high luminance (hereafter, a “bright” pixel), and low-intensity emissions may be represented by a pixel of low luminance (a “dim” pixel). Alternatively, the chromatic value of a pixel may be made to represent the intensity, color, or other characteristic of the detected emissions. Thus, an area of high-intensity emission may be displayed as a red pixel and an area of low-intensity emission as a blue pixel. As another example, detected emissions of one wavelength at a particular sub-area of the substrate may be represented as a red pixel, and emissions of a second wavelength detected at another sub-area may be represented by an adjacent blue pixel. Many other display schemes are known. Various techniques may be applied for identifying the data representing detected emissions and separating them from background information. For example, U.S. Pat. No. 6,090,555, and U.S. patent application Ser. No. 10/197,369, titled “System, Method, and Computer Program Product for Scanned Image Alignment” filed Jul. 17, 2002, which are both hereby incorporated by reference herein in their entireties for all purposes, describe various of these techniques. In a particular implementation, scanner 150 may identify one or more labeled targets. For instance, sample of a first target may be labeled with a first dye (an example of what may more generally be referred to hereafter as an “emission label”) that fluoresces at a particular characteristic frequency, or narrow band of frequencies, in response to an excitation source of a particular frequency. A second target may be labeled with a second dye that fluoresces at a different characteristic frequency. The excitation sources for the second dye may, but need not, have a different excitation frequency than the source that excites the first dye, e.g., the excitation sources could be the same, or different, lasers. The target samples may be mixed and applied to the probe arrays, and conditions may be created conducive to hybridization reactions, all in accordance with known techniques.

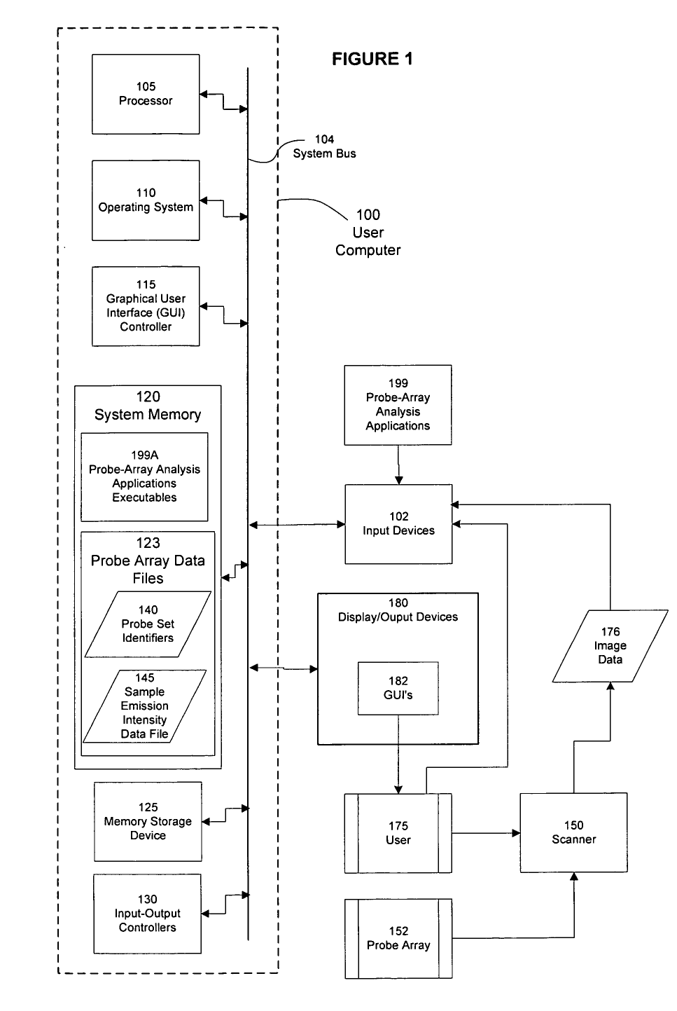

Probe Array 152: An illustrative example of probe array 152 is provided in FIG. 1. Descriptions of probe arrays are provided above with respect to “Nucleic Acid Probe arrays” and other related disclosure. In various implementations probe array 152 may be disposed in a cartridge or housing such as, for example, the GeneChip® probe array available from Affymetrix, Inc. of Santa Clara Calif.

For example, some implementations of probes disposed on probe array 152 may be designed to interrogate the sequence composition of DNA such as for instance, probes that interrogate single nucleotide polymorphisms (hereafter referred to as SNP's) or probes that interrogate the nucleotide composition at a specific sequence position. In some implementations, a process that is commonly referred to as polymerase chain reaction (hereafter referred to as PCR) may be used to amplify selected regions of DNA, where an individual probe is capable of detecting a specific nucleic acid at a specific sequence position within a PCR product or DNA sequence. In general, a group of probes, may be referred to as a probe set, where some probe-sets may for example comprise what is referred to at least one perfect match probe and at least one mismatch probe, where the perfect match probe is complementary to a sequence being interrogated and the mismatch probe differs in sequence composition with respect to the sequence to be interrogated at one or more sequence positions. Alternatively, some embodiments may include probe sets that include on perfect match probes.

For example, one possible embodiment may include genotyping probe sets, such as for instance probe sets designed to interrogate SNP's. In the present example each SNP may be represented by a collection of probe sets on probe array 152, each having a plurality of probes. Embodiments of probe array 152 may comprise between 1 and 10 probe sets for each SNP. In the present example, there may be 7 probe sets for each SNP. In the present example, each probe set may comprise 8 probes that correspond to a perfect match or PM probe for each of two alleles on the “coding” or sense strand, a mismatch or MM probe for each of 2 alleles, and the corresponding probes for the “non-coding” or anti-sense strand. In other words, for each SNP there may be a perfect match, a perfect mismatch, an antisense match and an antisense mismatch probe.

In some embodiments, probe sets for each SNP may vary from each other, such as for instance by the relative position of the polymorphic location in the probe sequence. For example, the polymorphic position may be the central position of the probe sequence. In the present example, the probe sequence may be 25 nucleotides in length and the polymorphic position may be the 13^{th} base in the sequence with 12 nucleotide sequence positions on either side. Probe sets may vary from one another with respect to the polymorphic position in the probe sequence, or the number of sequence positions that it may be offset from the 13^{th} center position. In the present example, the polymorphic position may be from 1 to 5 bases from the 13^{th} central position on either the 5′ or 3′ side of the probe sequence. The differences in sequence composition with respect to mismatch probes may be at the 13^{th} center position or at one or more other sequence positions in the probe sequence.

Continuing the above example, some embodiments of probe array 152 may include 7 probe sets for each SNP on each strand, where each probe set comprises 4 probes sometime referred to as probe cells. In the present example, each probe set varies the relative position of the polymorphic nucleic acid (i.e. one of the two nucleic acid possibilities associated with a biallelic SNP) with respect to the probe sequence. For instance, one probe set may include the polymorphic nucleic acid position at the center (i.e. 13^{th} sequence position of a 25 base probe) that may also be referred to as the 0 position. In addition, the six other probe sets may vary the polymorphic nucleic acid position at each of the following positions −4, −2, −1, +1, +3 and +4 relative to the 0 position, where the position value relates to the number of sequence positions away from the 0 position in the (−) direction and the (+) direction. In the present example, the (−) direction and the (+) direction are opposite of each other and could for instance be relative the 3′ or 5′ end of a sequence or other means of identifying sequence directionality.

Also, each embodiment of probe array 152 may include a plurality of probe sets each comprising a plurality of probes enabled to interrogate the nucleotide composition of each SNP position. Also, some embodiments include one or more probe sets enabled to interrogate sequence composition associated with a complementary sequence (i.e. complementary sequence by Watson-Crick base paring rules) region on each of the two strands of DNA, for example, the sense strand and the anti-sense strand of DNA.

Further details regarding the design and use of probes and probe sets are provided in U.S. Pat. No. 6,188,783; in PCT application Ser. No. PCT/US 01/02316, filed Jan. 24, 2001; in U.S. patent applications Ser. Nos. 09/721,042, 09/718,295, 09/745,965, and 09/764,324; U.S. patent application Ser. No. 10/681,773, titled “Methods for Genotyping Polymorphisms in Humans”, filed Oct. 7, 2003; and Ser. No. 10/891,260, titled “Methods for Genotyping Polymorphisms in Humans”, filed Jul. 13, 2004, all of which are hereby incorporated herein by reference in their entireties for all purposes.

Probe Set Identifiers 140: Probe-set identifiers typically come to the attention of a user, represented by user 175 of FIG. 1, as a result of experiments conducted on probe arrays. For example, user 175 may select probe-set identifiers that identify microarray probe sets capable of enabling detection of the expression of mRNA transcripts from corresponding genes or EST's of particular interest. As is well known in the relevant art, an EST is a fragment of a gene sequence that may not be fully characterized, whereas a gene sequence generally is complete and fully characterized. The word “gene” is used generally herein to refer both to full size genes of known sequence and to computationally predicted genes. In some implementations, the specific sequences detected by the arrays that represent these genes or EST's may be referred to as, “sequence information fragments (SIF's)” and may be recorded in what may be referred to as a “SIF file.” In particular implementations, a SIF is a portion of a consensus sequence that has been deemed to best represent the mRNA transcript from a given gene or EST. The consensus sequence may have been derived by comparing and clustering EST's, and possibly also by comparing the EST's to genomic sequence information. A SIF is a portion of the consensus sequence for which probes on the array are specifically designed. With respect to the operations of sequence data manager 323 of the particular implementation described herein, it is assumed with respect to some aspects that some microarray probe sets may be designed to detect the sequence composition of DNA from PCR amplified fragments.

As was described above, the term “probe set” refers in some implementations to one or more probes from an array of probes on a microarray. For example, in an Affymetrix® GeneChip® probe array, in which probes are synthesized on a substrate, a probe set may consist of 30 or 40 probes, half of which typically are controls. These probes collectively, or in various combinations of some or all of them, are deemed to be indicative of the expression of a gene or EST. In a spotted probe array, one or more spots may similarly constitute a “probe set.”

The term “probe-set identifiers” is used broadly herein in that a number of types of such identifiers are possible and may be included within the meaning of this term in various implementations. One type of probe-set identifier is a name, number, or other symbol that is assigned for the purpose of identifying a probe set. This name, number, or symbol may be arbitrarily assigned to the probe set by, for example, the manufacturer of the probe array. A user may select this type of probe-set identifier by, for example, highlighting or typing the name. Another type of probe-set identifier as intended herein is a graphical representation of a probe set. For example, dots may be displayed on a scatter plot or other diagram wherein each dot represents a probe set, as described for example in U.S. Pat. No. 6,420,108, which is hereby incorporated herein in its entirety for all purposes. Typically, the dot's placement on the plot represents the intensity of the signal from hybridized, tagged, targets (as described in greater detail below) in one or more experiments. In these cases, a user may select a probe-set identifier by clicking on, drawing a loop around, or otherwise selecting one or more of the dots. In another example, user 175 may select a probe-set identifier by selecting a row or column in a table or spreadsheet that correlates probe sets with accession numbers and other genomic information.

Yet another type of probe-set identifier, as that term is used herein, includes a nucleotide or amino acid sequence. For example, it is illustratively assumed that a particular SIF is a unique sequence of 500 bases that is a portion of a consensus sequence or exemplar sequence gleaned from EST and/or genomic sequence information. It further is assumed that one or more probe sets are designed to represent the SIF. A user who specifies all or part of the 500-base sequence thus may be considered to have specified all or some of the corresponding probe sets.

As a further example with respect to a particular implementation, a user may specify a portion of the 500-base sequence noted above, which may be unique to that SIF, or, alternatively, may also identify another SIF, EST, cluster of EST's, consensus sequence, and/or gene or protein. The user thus specifies a probe-set identifier for one or more genes or EST's. In another variation, it is illustratively assumed that a particular SIF is a portion of a particular consensus sequence. It is further assumed that a user specifies a portion of the consensus sequence that is not included in the SIF but that is unique to the consensus sequence or the gene or EST's the consensus sequence is intended to represent. In that case, the sequence specified by the user is a probe-set identifier that identifies the probe set corresponding to the SIF, even though the user-specified sequence is not included in the SIF. Parallel cases are possible with respect to user specifications of partial sequences of EST's and genes or EST's, as those skilled in the relevant art will now appreciate.

A further example of a probe-set identifier is an accession number of a gene or EST. Gene and EST accession numbers are publicly available. A probe set may therefore be identified by the accession number or numbers of one or more EST's and/or genes corresponding to the probe set. The correspondence between a probe set and EST's or genes may be maintained in a suitable database from which the correspondence may be provided to the user. Similarly, gene fragments or sequences other than EST's may be mapped (e.g., by reference to a suitable database) to corresponding genes or EST's for the purpose of using their publicly available accession numbers as probe-set identifiers. For example, a user may be interested in product or genomic information related to a particular SIF that is derived from EST-1 and EST-2. The user may be provided with the correspondence between that SIF (or part or all of the sequence of the SIF) and EST-1 or EST-2, or both. To obtain product or genomic data related to the SIF, or a partial sequence of it, the user may select the accession numbers of EST-1, EST-2, or both.

Additional examples of probe-set identifiers include one or more terms that may be associated with the annotation of one or more gene or EST sequences, where the gene or EST sequences may be associated with one or more probe sets. For convenience, such terms may hereafter be referred to as “annotation terms” and will be understood to potentially include, in various implementations, one or more words, graphical elements, characters, or other representational forms that provide information that typically is biologically relevant to or related to the gene or EST sequence. Associations between the probe-set identifier terms and gene or EST sequences may be stored in a database such as a local genomic database, or they may be transferred from one or more remote databases. Examples of such terms associated with annotations include those of molecular function (e.g. transcription initiation), cellular location (e.g. nuclear membrane), biological process (e.g. immune response), tissue type (e.g. kidney), or other annotation terms known to those in the relevant art.

In some embodiments, a relevant example of a probe set identifier may include the SNP ID. It is well known to those skilled in the related art that the most common type of human genetic variation is the Single Nucleotide polymorphism, commonly referred to as a SNP, a position at which two alternative bases occur at appreciable frequency, say for instance >1% in the human population. Each SNP may be identified by a characteristic identifier. This identifier may for instance represent the position of the SNP or any other random number that would help identify the SNP. Alternatively the probe set identifier for a SNP, namely the SNP ID may provide a short description of the SNP. For example, the user may provide a SNP ID 110373 as a probe set identifier to obtain product or genomic data associated with the SNP ID.

Probe-Array Analysis Applications 199: Generally, a human being may inspect a printed or displayed image constructed from the data in an image file and may identify those cells that are bright or dim, or are otherwise identified by a pixel characteristic (such as color). However, it frequently is desirable to provide this information in an automated, quantifiable, and repeatable way that is compatible with various image processing and/or analysis techniques. For example, the information may be provided for processing by a computer application that associates the locations where hybridized targets were detected with known locations where probes of known identities were synthesized or deposited. Other methods include tagging individual synthesis or support substrates (such as beads) using chemical, biological, electromagnetic transducers or transmitters, and other identifiers. Information such as the nucleotide or monomer sequence of target DNA or RNA may then be deduced. Techniques for making these deductions are described, for example, in U.S. Pat. No. 5,733,729, which hereby is incorporated by reference in its entirety for all purposes, and in U.S. Pat. No. 5,837,832, noted and incorporated above.

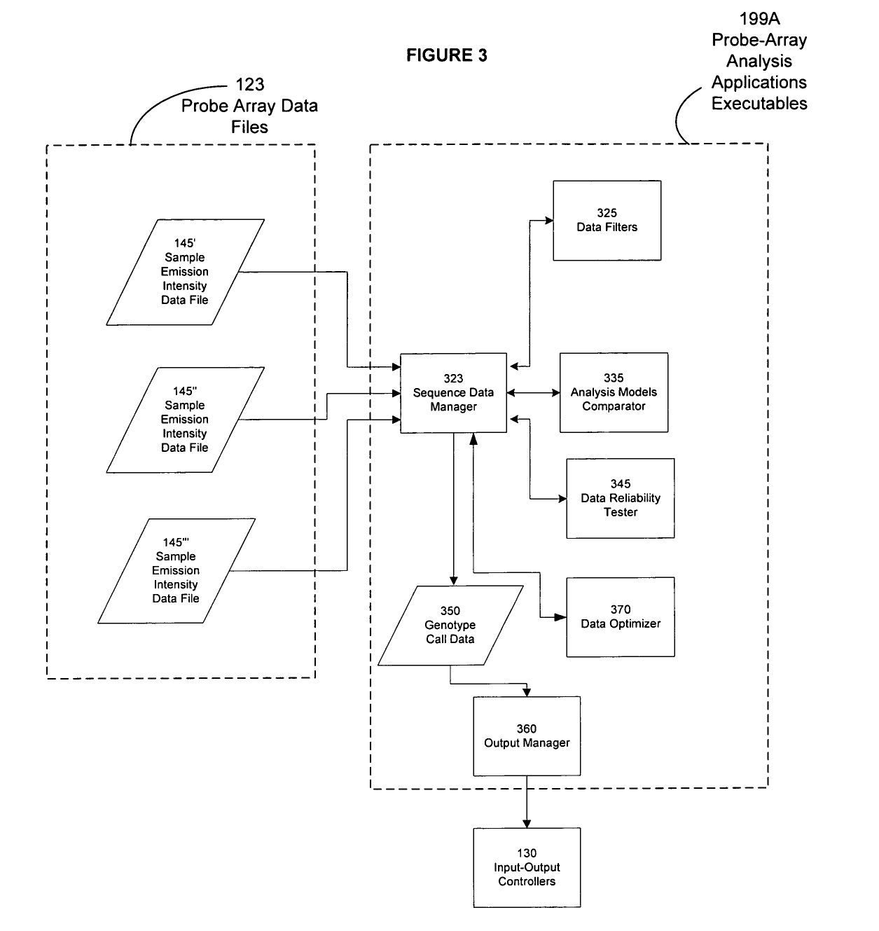

A variety of computer software applications are commercially available for controlling scanners (and other instruments related to the hybridization process, such as hybridization chambers), and for acquiring and processing the image files provided by the scanners. Examples are the Jaguar™ application from Affymetrix, Inc., aspects of which are described in PCT Application PCT/US 01/26390 and in U.S. patent applications, Ser. Nos. 09/681,819, 09/682,071, 09/682,074, 09/682,076, and 10/197,369, and the Microarray Suite application from Affymetrix, aspects of which are described in U.S. Provisional Patent Applications, Ser. Nos. 60/220,587, 60/220,645 and 60/312,906, and in U.S. patent application Ser. No. 10/219,882; and the GeneChip® Operating Software (hereafter referred to as GCOS) aspects of which are described in U.S. Provisional Application Ser. Nos. 60/442,684, titled “System, Method and Computer Software for Instrument Control and Data Acquisition, Analysis, Management and Storage”, filed Jan. 24, 2003, and 60/483,812, titled “System, Method and Computer Software for Instrument Control, Data Acquisition and Analysis”, filed Jun. 30, 2003, all of which are hereby incorporated herein by reference in their entireties for all purposes. For example, image data in image data file 176 may be operated upon to generate intermediate results such as so-called cell intensity files (*.cel) and chip files (*.chp), generated by Microarray Suite or GCOS, or spot files (*.spt) generated by Jaguar™ software. For convenience, the terms “file” or “data structure” may be used herein to refer to the organization of data, or the data itself generated or used by executables 199A and executable counterparts of other applications. However, it will be understood that any of a variety of alternative techniques known in the relevant art for storing, conveying, and/or manipulating data may be employed, and that the terms “file” and “data structure” therefore are to be interpreted broadly. In the illustrative case in which image data file 176 is derived from a GeneChip® probe array, and in which Microarray Suite or GCOS may generate one or more data files contained in probe array data files 123. FIG. 3 further illustrates an example of data files 123 that may include sample emission intensity data file 145′, 145″, and 145′″. Each of data files 145 may contain emission intensity data for each probe feature disposed upon a probe array. In the present example data file 145′ may correspond to a particular probe array type where an experimental sample has been tested. Additionally, data file 145″ and 145′″ may correspond to the same probe array type where different experimental samples have been used that may allow for the comparison between experimental samples. Those of ordinary skill in the related art will appreciate that each of files 145 may include one or more data files that may correspond to one or more experimental samples.

Files 145 may contain, for each probe feature scanned by scanner 150, a single value representative of the intensities of pixels measured by scanner 150 for that probe feature. Thus, this value is a measure of the abundance of tagged cRNA's present in the target that hybridized to the corresponding probe feature. Many such cRNA's may be present in each probe feature, as a probe feature on a GeneChip® probe array may include, for example, millions of oligonucleotides designed to detect the cRNA's. The resulting data stored in the chip file may include degrees of hybridization, absolute and/or differential (over two or more experiments) expression, genotype comparisons, detection of polymorphisms and mutations, and other analytical results. In another example, in which executables 199A includes image data from a spotted probe array, the resulting spot file includes the intensities of labeled targets that hybridized to probes in the array. Further details regarding cell files, chip files, and spot files are provided in U.S. Provisional Patent Application Nos. 60/220,645, 60/220,587, and 60/226,999, incorporated by reference above.

In the present example, in which executables 199A include Affymetrix® Microarray Suite or GCOS, the chip file is derived from analysis of the cell file combined in some cases with information derived from library files. Laboratory or experimental data may also be provided to the software for inclusion in the chip file. For example, an experimenter and/or automated data input devices or programs may provide data related to the design or conduct of experiments. As a non-limiting example, the experimenter may specify an Affymetrix catalogue or custom chip type (e.g., Human Genome U95Av2 chip) either by selecting from a predetermined list presented by Microarray Suite or GCOS or by scanning a bar code related to a chip to read its type. Also, this information may be automatically read. For example, a bar code (or other machine-readable information such as may be stored on a magnetic strip, in memory devices of a radio transmitting module, or stored and read in accordance with any of a variety of other known techniques) may be affixed to the probe array, a cartridge, or other housing or substrate coupled to or otherwise associated with the array. The machine-readable information may automatically be read by a device (e.g., a 1-D or 2-D bar code reader) incorporated within the scanner, an autoloader associated with the scanner, an autoloader movable between the scanner and other instruments, and so on. In any of these cases, Microarray Suite may associate the chip type, or other identifier, with various scanning parameters stored in data tables. The scanning parameters may include, for example, the area of the chip that is to be scanned, the starting place for a scan, the location of chrome borders on the chip used for auto-focusing, the speed of the scan, a number of scan repetitions, the wavelength or intensity of laser light to be used in reading the chip, and so on. Rather than storing this data in data tables, some or all of it may be included in the machine-readable information coupled or associated with the probe arrays. Other experimental or laboratory data may include, for example, the name of the experimenter, the dates on which various experiments were conducted, the equipment used, the types of fluorescent dyes used as labels, protocols followed, and numerous other attributes of experiments.

As noted, executables 199A may apply some of this data in the generation of intermediate results. For example, information about the dyes may be incorporated into determinations of relative expression. Other data, such as the name of the experimenter, may be processed by executables 199A or may simply be preserved and stored in files or other data structures. Any of these data may be provided, for example over a network, to a laboratory information management server computer, configured to manage information from large numbers of experiments. A data analysis program may also generate various types of plots, graphs, tables, and other tabular and/or graphical representations of analytical data. As will be appreciated by those skilled in the relevant art, the preceding and following descriptions of files generated by executables 199A are exemplary only, and the data described, and other data, may be processed, combined, arranged, and/or presented in many other ways.

The processed image files produced by these applications often are further processed to extract additional data. In particular, data-mining software applications often are used for supplemental identification and analysis of biologically interesting patterns or degrees of hybridization of probe sets. An example of a software application of this type is the Affymetrix® Data Mining Tool, described in U.S. patent application, Ser. No. 09/683,980, and Affymetrix® GeneChip® Data Analysis Software (hereafter referred to as GDAS), described in U.S. Provisional Patent Application Ser. No. 60/408,848, titled “System, Method, and Computer Software Product for Determination and Comparison of Biological Sequence Composition”, filed Sep. 6, 2002; and U.S. patent application Attorney Ser. No. 10/657,481, titled “System, Method, and Computer Software Product For Analysis And Display of Genotyping, Annotation, and Related Information”, filed Sep. 9, 2003, each of which is hereby incorporated herein by reference in its entireties for all purposes. Software applications also are available for storing and managing the enormous amounts of data that often are generated by probe-array experiments and by the image-processing and data-mining software noted above. An example of these data-management software applications is the Affymetrix® Laboratory Information Management System (LIMS). In addition, various proprietary databases accessed by database management software, such as the Affymetrix® EASI (Expression Analysis Sequence Information) database and database software, provide researchers with associations between probe sets and gene or EST identifiers.

For convenience of reference, these types of computer software applications (i.e., for acquiring and processing image files, data mining, data management, and various database and other applications related to probe-array analysis) are generally and collectively represented in FIG. 1 as probe-array analysis applications 199. FIG. 1 illustratively shows applications 199 stored for execution (as executable code 199A corresponding to applications 199) in system memory 120 of user computer 100.

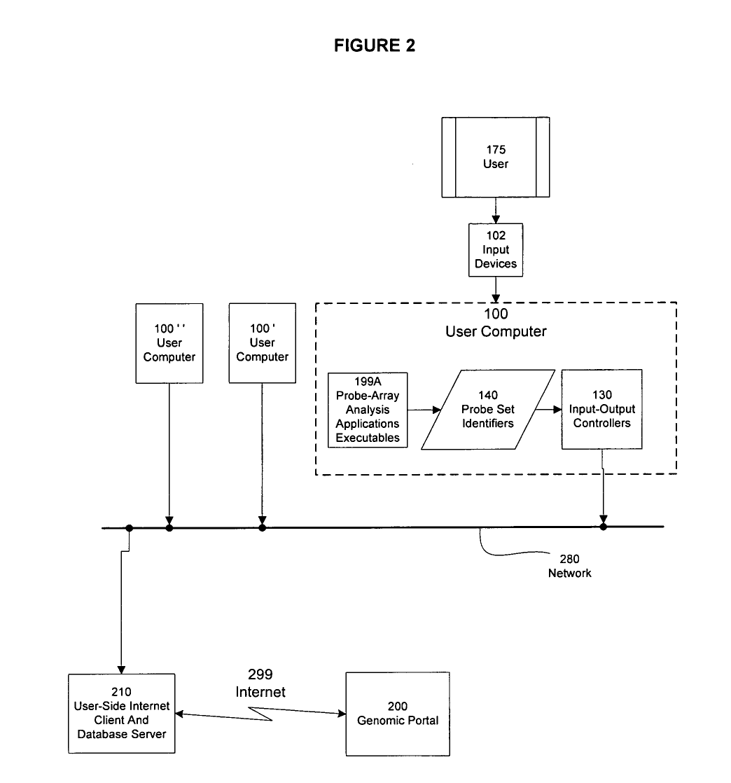

As will be appreciated by those skilled in the relevant art, it is not necessary that applications 199 be stored on and/or executed from computer 100; rather, some or all of applications 199 may be stored on and/or executed from an applications server or other computer platform to which computer 100 is connected in a network. For example, it may be particularly advantageous for applications involving the manipulation of large databases to be executed from a database server such as user-side internet client and database server 210 of FIG. 2. Alternatively, LIMS, DMT, and/or other applications may be executed from computer 100. But some or all of the databases upon which those applications operate may be stored for common access on server 210 (perhaps together with a database management program, such as the Oracle® 8.0.5 database management system from Oracle Corporation). Such networked arrangements may be implemented in accordance with known techniques using commercially available hardware and software, such as those available for implementing a local-area network or wide-area network. A local network is represented as network 280 by the connection of user computer 100 to database server 210 (and to a user-side Internet client, which is illustrated in FIG. 2 as the same computer but need not be). The connections of network 280 could include a network cable, wireless network, or other means of networking known to those in the related art. Similarly, scanner 150 (or multiple scanners) may be made available to a network of users over a network cable both for purposes of controlling scanner 150 and for receiving data input from it.

In some implementations, it may be convenient for user 175 to group probe-set identifiers for batch transfer of information or to otherwise analyze or process groups of probe sets together. For example, as described below, user 175 may wish to obtain annotation information related to one or more probe sets identified by their respective probe set identifiers 140. Rather than obtaining this information serially, user 175 may group probe sets together for batch processing. Various known techniques may be employed for associating probe set identifiers 140, or data related to those identifiers, together. For instance, user 175 may generate a tab delimited *.txt file including a list of probe set identifiers 140 for batch processing. This file or another file or data structure for providing a batch of data (hereafter referred to for convenience simply as a “batch file”), may be any kind of list, text, data structure, or other collection of data in any format. The batch file may also specify what kind of information user 175 wishes to obtain with respect to all, or any combination of, the identified probe sets. In some implementations, user 175 may specify a name or other user-specified identifier to represent the group of probe-set identifiers specified in the text file or otherwise specified by user 175. This user-specified identifier may be stored by one of executables 199A, so that user 175 may employ it in future operations rather than providing the associated probe-set identifiers in a text file or other format. Thus, for example, user 175 may formulate one or more queries associated with a particular user-specified identifier, resulting in a batch transfer of information from portal 200 to user 175 related to the probe-set identifiers that user 175 has associated with the user-specified identifier. Alternatively, user 175 may initiate a batch transfer by providing the text file of probe-set identifiers. In any of these cases, user 175 may provide information, such as laboratory or experimental information, related to a number of probe sets by a batch operation rather than serial ones. The probe sets may be grouped by experiments, by similarity of probe sets (e.g., probe sets representing genes having similar annotations, such as related to transcription regulation), or any other type of grouping. For example, user 175 may assign a user-specified identifier (e.g., “experiments of January 1”) to a series of experiments and submit probe-set identifiers in user-selected categories (e.g., identifying probe sets that were up-regulated by a specified amount).

Similarly, user 175 may use probe set identifiers 140 for the design of custom probe arrays. User 175 may want to use probe arrays with a particular combination of probe sets disposed upon them that may not be available as a commercial product. Additionally, a user may wish to use probe sets that are not available. In both cases the user may submit a plurality of probe set identifiers and other selected specifications for the custom production of probe sets, and/or probe arrays. User 175 may electronically submit probe set identifiers individually or by batch transfer as previously described. The methods electronic submission could include submission by e-mail, or other methods of electronic submission known to those of ordinary skill in the related art. One such example is illustrated in FIG. 2 where the user may submit the probe set identifiers via Internet 299 to genomic portal 200. Portal 200 may interactively provide the user with information that could include a confirmation that the plurality of probe set identifiers had been received, expected shipping dates, price quotes, or other information that might be of interest to the user. In the present example, portal 200 is specifically enabled to receive a plurality of probe set identifiers for probe array design. Portal 200 could for instance be a web portal provided by Affymetrix®, Inc.

Further details regarding the submission of probe set identifiers for custom array design are described in U.S. Provisional Patent Application 60/310,298, and U.S. patent application Ser. No. 10/036,559, each of which is hereby incorporated by reference herein in their entireties for all purposes.

Sequence Data Manager 323: Another element of probe array analysis executables 199A may include sequence data manager 323. In one embodiment sequence data manager 323 may manage the functions of analyzing the emission intensity values contained within probe array data files 123, illustrated in FIG. 3 as data file 145′, data file 145″, and data file 145′″. For example, each of data files 145 includes emission intensity data obtained from a probe array experiment, in particular what may be referred to as a genotyping experiment such as an experiment based upon the analysis of DNA sequence. In the present example, data manager 323 may concurrently analyze a plurality of data files 145 associated with samples that could, for instance, include 200 or more samples.

In some embodiments, data manager 323 may employ genotyping algorithms that identify the composition of nucleic acids of a selected DNA sequence, single nucleotide polymorphisms (hereafter referred to as SNP's), or other features related to aspects of genomic sequence. For example, one type of algorithm could include the CustomSeq™ algorithm from Affymetrix, Inc. The CustomSeq™ algorithm may be used to determine nucleic acid composition for selected sequence regions or positions of a DNA sequence. For example, an algorithm may analyze emission intensity data values associated with a plurality of probe sets directed to a target sequence, where the plurality of probe sets are disposed on probe arrays designed to interrogate genomic DNA or other type of sequences.

Data Filters 325: In some embodiments, manager 323 may employ a genotyping algorithm for the analysis of the emission intensity data values that comprises a number of steps that may, for instance, be implemented by one or more hardware or software elements. For example, manager 323 may initially employ data filters 325 to identify unreliable data or adjust what is referred to as the variance of the emission intensities that may approach the limits of detection of a scanner instrument. The term “variance” as used herein generally refers to a value that is a measure of the dispersion of data. For example, it will be appreciated by those skilled in the relevant art that, variance may be defined as the mean of the square of the differences between the samples and can be mathematically represented as:

$$Equation \text{-} 1 \text{:} {σ}^{2} = \frac{\sum {( X - \overline{X} )}^{2}}{n - 1}$$

where, X is equal to a particular value that could for instance be an emission intensity value for a probe feature.
\overline{X} is equal to the mean of all the values
n is equal to the total number of values.

In some embodiments, data filters 325 may analyze emission intensity data values that correspond to a plurality probe sets directed to a region of DNA sequence or position of SNP in an associated sample to determine whether the emission intensity data is reliable. For example, if data filters 325 determines that the data is not reliable, filters 325 will assign a “no call” (n) genotype call associated with the unreliable data or make an adjustment to one or more variance values. For example, data filters 325 may analyze or pre-filter the emission intensity data based upon categories of signal characteristics that could, for instance include no signal, weak signal, saturated signal, or high signal to noise ratio characteristics. Also in the present example, if data filters 325 determines that a sequence position or target is ruled as a no call (n), then that information may be recorded as the genotype call for the target in genotype call data 350. Further examples of categories of signal characteristics are presented in greater detail below.

Data filters 325 may determine that the emission intensity data fits the “no signal” category if the data does not meet a threshold value associated with what may be referred to as the mean intensity value. For example, each probe feature of each probe set may have a mean intensity value associated with it that may, for instance, be defined as the mean of the emission intensity values for the pixels associated with the probe feature defined by the boundaries of a “grid” (sometimes referred to as a cell). The threshold value could include a pre-defined value or user-selectable value, where a pre-defined value could include a value that is within two standard deviations of zero. The term “standard deviation” as used herein generally refers to a value that is the square root of the variance. If, for example, the mean intensity value for any probe feature of a probe set is below the threshold value then the call assigned to the corresponding sequence position or target will be no call (n). Otherwise the criteria have been satisfied for the category and a call may not be assigned by filters 325.

Data filters 325 may determine that the emission intensity data fits the “weak signal” category if the data does not meet a threshold value for what may be referred to as the highest mean intensity value. For example, the highest mean intensity value may be defined as the mean intensity value for a probe feature that is higher than all other mean intensity values of probe features of the probe set. The threshold value could include a pre-defined or user-selectable value such as a value equal to a 20 fold decrease from the average highest mean intensities for all probe sets from the same strand (i.e. sense or anti-sense strands). In the present example, if the highest mean intensity value for a probe set is below the threshold value then data filters 325 will assign the sequence position or target as a no call genotype call. Otherwise the criteria have been satisfied for the category and a call may not be assigned by data filters 325.

Data filters 325 may determine that the emission intensity data fits the “saturated signal” category if the emission intensity data associated with one or more of the probe features of a probe set exceeds a threshold value. For example, a plurality of probe features of a probe set may need to exceed the threshold value in order for a no call assignment to be made. In the present example, the threshold value could include a pre-defined or user-selectable value such as a value that is two standard deviations below 43,000. The number of 43,000 is used in the present example as a representation of an emission intensity value that may be at the limit of detection for scanning 150. But those of ordinary skill in the related art will appreciate that other values may be used that are representative of the detection limits of particular systems. The standard deviation value may be the same as that used for the “no signal” category, or may be different being derived from another set of emission intensity values. Also, data filters 325 may employ a second criterion such as a number of the probe features that exceed the threshold value in order to assign a no call to the sequence position or target. For example, a sequence position or target sequence may be located on a single chromosome, where the single chromosome may be referred to as being in a haploid state (i.e. generally a haploid state refers to the presence of a single chromosome, and a diploid state refers to a pair of similar chromosomes). If two or more probe features of a probe set have mean intensity values greater than the threshold value then the sequence position or target sequence is assigned as a no call. Also in the present example, if the sequence position or target sequence is located on a pair of chromosomes that may be referred to as a diploid state, then data filters 325 may require that three or more features must exceed the threshold value for a no call assignment to be made by data filters 325.

Data filters 325 may determine that the emission intensity data fits the “signal to noise ratio” category if the emission intensity data associated with one or more of the probe features of a probe set exceeds a threshold value. The term “signal to noise ratio” as used herein generally refers to the ratio of emission intensity values from the signal generated from hybridized probes to the emission intensity values from what is referred to as noise. Noise may include fluorescent emissions generated from residual unbound sample, the non-specific binding of sample to probe features, electronic noise from detectors sometimes referred to as “dark current”, or other sources or noise known in the art. The threshold may include a pre-defined or use selected value such as, for instance 20. In some implementations, if the signal to noise ratio exceeds the threshold value, filters 325 may adjust one or more parameters such as, for instance variance, so that the signal to noise ration is equal to the threshold value. For example, if the signal to noise ratio for all probe sets of a given sample is greater than 20, then the variance for all probe sets of the sample may be set at so that the signal to noise ratio is equal to 20. In an alternative example, the signal to noise ratio within a probe set, or the one or more probe sets that correspond to a sequence position may be greater than the threshold value. In such an example the variance that corresponds to the one or more probe sets may be set so that the signal to noise ratio of the one or more probe sets is equal to the threshold value.

Analysis Model Comparator 335: Sequence data manager 323 may then forward the filtered emission intensity data to genotype call generator 335 to perform the next steps. The processes performed by comparator 335 may be based, at least in part, upon models developed to specify the presence or absence of specific nucleic acids in each sequence position of a selected DNA sequence. Different sets of models may be applied to the data based upon different assumptions. The assumptions may be based upon what may be referred to as an even background or uneven background that will be explained in more detail below.

In one embodiment, comparator 335 may calculate what may be referred to as a maximum likelihood functions associated with each genotype state in order to determine the most likely genotype call. For example, the likelihood may be determined for both the sense and the anti-sense strands together at different states in order to determine the model that best fits the data. The likelihood and log-likelihood functions are the basis for deriving estimators for the data. Both these functions have a common maximum point. The maximum point known to those skilled in the relevant art as the Maximum Likelihood estimate (MLE) may be defined as the ‘most likely’ value relative to the others. Therefore the state with the maximum likelihood may be the model that best fits the state. For example, null, AA, BB, and AB may be the four models assigned to the 4 states for example, Null, homozygous state (AA and BB) and heterozygous state (AB), where, for example, both A and B may refer to alleles in a biallelic SNP.

Comparator 335 may calculate the maximum likelihood for each of the four models using emission intensity data from a plurality of probe sets for each sequence position or target sequence from files 145. Each of the models comprises a set of assumptions that are true if the data fits the model. For example, a probe set may be comprised of four cells that may also be referred to as a quartet where each of the cells is independent of each other. In the present example, each of the models assumes the pixel signal intensities for any given cell are independent, identically distributed, normal random variables. Further, each of the models may also assume that the sense and anti-sense sequences or strands are independent of each other, and the cells referred to as foreground cells in each of the models have a mean intensity value above some threshold value as determined previously by data filters 325. Similarly the cells referred to as the background cells in each of the models include mean intensity value below a threshold value. Additionally, for each of the models it may be assumed that both the foreground and the background cells are evenly distributed distribution, in other words, a all fore ground cells have the same distribution (i.e. a Gaussian distribution), and all of the background cells have the same distribution (i.e. a Gaussian distribution).

Comparator 335 may perform calculations employing the emission intensities from each pixel for each cell of each probe set. For example, the calculations may include an observed mean ^{μ}ₖ, observed variance ⁹⁴ ^{²}ₖ, estimated mean ^{\hat{μ}}ₖ, estimated variance \hat{σ}^{²}ₖ, and number of observations—nₖ that may, for instance, include the number of pixels in a cell. For both the observed and the estimated conditions, k includes the number of cells being considered. In the present example, each probe set or quartet may comprise 4 probe cells (i.e. probe features) and therefore k=1, 2, 3, 4. It will be appreciated by those skilled in the related art that a log-likelihood of the maximum likelihood function may help to link the data, unknown model parameters and assumptions and hence allows rigorous, statistical inferences. Therefore, it will be known to those skilled in the relevant art that, in order to minimize the estimation error, an explicit log-likelihood function for a probe set may be given by,

$$ln ( L ) = - \frac{1}{2} {\sum }_{k = 1}^{4} {n}_{k} [ ln ( 2 π {\hat{σ}}_{k}^{2} ) + \frac{{σ}_{k}^{2} + ( {μ}_{k} - {\hat{μ}}_{k} )}{{\hat{σ}}_{k}^{2}} ] \qquad Equation \text{-} 2$$

where, ⁿₖ is the number of pixels observed in the feature k,

It will be appreciated by those of ordinary skill in the relevant art that the assumptions for an even background may be based, at least in part, upon what is referred to as the central limit theorem that generally allows making inferences about population means using the normal distribution no matter what the distribution of the population being sampled from. For example, each probe feature or cell of a probe set comprises a plurality of probes with identical sequence composition that may be relatively independent in their chance of binding a labeled target. Therefore as will be appreciated by those of ordinary skill in the related art, the overall emission intensity of the feature should be normally distributed (i.e. the probes have an equal chance of binding to the target molecules in the sample). Accordingly the central limit theorem may be applied to different models mentioned, for example, Null model, homozygous model, heterozygous model in order to obtain the corresponding equations.

Null Model: The maximum likelihood estimators for the null model where all the cells are assumed as background and evenly distributed may have a mean and variance of

$$\hat{μ} ≡ {\hat{μ}}_{1} = {\hat{μ}}_{2} = {\hat{μ}}_{3} = {\hat{μ}}_{4} = \frac{{\sum }_{k = 1}^{4} {n}_{k} {μ}_{k}}{{\sum }_{k = 1}^{4} {n}_{k}} \\ {\hat{σ}}_{1}^{2} = {\hat{σ}}_{2}^{2} = {\hat{σ}}_{3}^{2} = {\hat{σ}}_{4}^{2} = \frac{{\sum }_{k = 1}^{4} {n}_{k} [ {σ}_{k}^{2} + {μ}_{k}^{2} ]}{{\sum }_{k = 1}^{4} {n}_{k}} - {\hat{μ}}^{2} \qquad Equation \text{-} 3$$

Homozygote Model: The homozygote models for AA and BB may be similar to the no call model, but with slightly different assumptions in regards to their background. For example, the maximum likelihood estimators for the AA model wherein cell 1 of each probe set may be associated with the perfect match probe for the A allele and considered foreground, and may include a mean and variance of
\hat{μ}₁=μ_{1 \hat{σ}}₁²=σ₁² Equation-4:

Similarly the mean and variance of probe set cells 2, 3 and 4 may be considered as background and evenly distributed, where the mean and variance of probe set cells 2, 3, and 4 may be given by,

$$Equation 4.1 \text{:} {\hat{μ}}_{2} = {\hat{μ}}_{3} = {\hat{μ}}_{4} = \frac{{{\sum }_{k = 2}}^{4} {n}_{k} {μ}_{k}}{{{\sum }_{k = 2}}^{4} {n}_{k}}$$

$${\hat{σ}}_{2}^{2} = {\hat{σ}}_{3}^{2} = {\hat{σ}}_{4}^{2} = \frac{{\sum }_{k = 2}^{4} {n}_{k} [ {σ}_{k}^{2} + {( {\hat{μ}}_{k} - {μ}_{k} )}^{2} ]}{{\sum }_{k = 2}^{4} {n}_{k}}$$

The same likelihood estimation process may apply to the other homozygous model, for example, the BB model wherein probe set cell 3 may be associated with the perfect match probe for the B allele and considered foreground while the other three probe set cells, for example, 1, 2 and 4 are assumed as background with an even distribution. Hence the mean and variance for the BB model may be given by:
\hat{μ}₃=μ_{3 \hat{σ}}₃²=σ₃² Equation-5

While the mean and variance for the other cells i.e. 1, 2, and 4 may be given by

$${\hat{μ}}_{1} = {\hat{μ}}_{2} = {\hat{μ}}_{4} = \frac{{\sum }_{k \neq 3} {n}_{k} {μ}_{k}}{{\sum }_{k \neq 3} {n}_{k}} \\ {\hat{σ}}_{1}^{2} = {\hat{σ}}_{2}^{2} = {\hat{σ}}_{4}^{2} = \frac{{\sum }_{k \neq 3}^{4} {n}_{k} [ {σ}_{k}^{2} + {( {\hat{μ}}_{k} - {μ}_{k} )}^{2} ]}{{\sum }_{k \neq 3}^{4} {n}_{k}} \qquad Equation \text{-} 5.1$$

In the present example, if the estimated mean for the model is less than the estimated mean of the background, then the likelihood is set to the no call model.

Heterozygote Model: The heterozygote model, for example AB may be statistically approached with a different assumption in regards to their backgrounds. In the AB model, probe set cells 1 and 3 as stated above are associated with the perfect match probes for the A and B alleles respectively and are assumed as foreground and evenly distributed while probe set cells 2 and 4 are associated with the mismatch probes and assumed as background and evenly distributed. Therefore maximum likelihood estimators for the AB model may be given as,

For Probe Set Cells 1 and 3:

$$\begin{matrix}{\hat{μ}}_{1} = {\hat{μ}}_{3} = \frac{{n}_{1} {μ}_{1} + {n}_{3} {μ}_{3}}{{n}_{1} + {n}_{3}} \\ {\hat{σ}}_{1}^{2} = {\hat{σ}}_{3}^{2} = \frac{{n}_{1} [ {σ}_{1}^{2} + {( {\hat{μ}}_{1} - {μ}_{1} )}^{2} ] + {n}_{3} [ {σ}_{3}^{2} + {( {\hat{μ}}_{3} - {μ}_{3} )}^{2} ]}{{n}_{1} + {n}_{3}}\end{matrix} \qquad Equation \text{-} 6$$

For Probe Set Cells 2 and 4:

$$\begin{matrix}{\hat{μ}}_{2} = {\hat{μ}}_{4} = \frac{{n}_{2} {μ}_{2} + {n}_{4} {μ}_{4}}{{n}_{2} + {n}_{4}} \\ {\hat{σ}}_{1}^{2} = {\hat{σ}}_{3}^{2} = \frac{{n}_{2} [ {σ}_{2}^{2} + {( {\hat{μ}}_{2} - {μ}_{2} )}^{2} ] + {n}_{4} [ {σ}_{4}^{2} + {( {\hat{μ}}_{4} - {μ}_{4} )}^{2} ]}{{n}_{2} + {n}_{4}}\end{matrix} \qquad Equation \text{-} 6.1$$

The log-likelihood functions may have a single mode or maximum point and no local optima and therefore maximizing the likelihood functions can get the best fit model with optimal outcome. Hence maximum likelihood estimators may be obtained for all parameters in different states.

For example comparator 335 may calculate 4 log-likelihood (Equation 2) values for each probe set or quartet using the calculated (from Equations 2-6) observed mean, estimated mean, and variance values, and maximum likelihood estimators. In the present example, the four log likelihoods for each probe set or quartet with four states may be obtained such that,
L(1)=L(Null),L(2)=L(AA),L(3)=L(BB),L(4)=L(AB)

Comparator 335 may employ statistical tests for each model and applied to a plurality of probe sets. For instance, statistics (S) for a model for example, model ‘m’ may be defined as:
S(m)=L(m)−max{L(k)}_{k≠m} where k,m=1,2,3,4. Equation-7

For example, comparator 335 may derive vectors of the statistics for the model m, using values from multiple probe sets. In the present example, values associated with detected intensities from 14 probe sets or quartets from both the sense and the antisense strands (i.e. 7 probe sets on each strand) for each target sequence that could include a SNP sequence thereby giving the equation:
{S^{q1}(m),S^{q2}(m), . . . ,S^{qi}(m)} Equation-8

Where m=1,2,3,4 (i.e. models 1-4) and qᵢ is the probe set indices, such as in the present example i=14 because of the 14 probe sets.

In some embodiments, statistical models may not make assumptions about the distribution of data. For example comparator 335 may employ alternative non-parametric statistical tests such as for instance, Chi-Square, Fisher exact probability test, Mann-Whitney test may be used for the statistical testing genotyping algorithms.

Comparator must then determine the best fitting model to the emission intensity data from the plurality of probe sets or quartets under consideration. It will be appreciated by those skilled in the related art that for a model m, if ^{S}^{^{qi}}^{(m)}>0, then the model m would be the best fitting model for that probe set or quartet. Where ^{S}^{^{qi}}^{(m)} is the symbolic representation of the statistics of the probe set or quartet qi of a given model m, and therefore the inference is that the emission intensity data associated with the probe set or quartet qi supports the model.

Alternatively if ^{S}^{^{qi}}^{(m)}<0, then the model m would not be the best fitting model for that probe set or quartet and the inference would be that the emission intensity data associated with the probe set or quartet qᵢ does not support the given model.

Therefore a non-parametric statistical test such as the one sided Wilcoxon signed rank test may be applied to the vectors of the statistics as described with respect to Equation 7, for all the models.

For example, comparator 335 may employ the Wilcoxon signed rank test to statistically determine how many probe sets support a given model. In the present example, the Wilcoxon signed rank test may be employed as an alternative to what is referred to as the t-test, which is a standard method used to test the difference between population means. In certain cases, for instance, if the population is not normally distributed, for instance in case of small samples, then the t-test may not produce a valid result. Therefore the application of a non-parametric statistical approach generally produces a more desirable result. In the present example comparator 335 may apply the Wilcoxon test to the 4 models and 14 probe sets as described with respect to Equations 3 through 6 based, at least in part, upon the null hypothesis as described in Equation 9.
H0: median {^{S}^{^{qi}}^{(m)}>0}>0 (vs) H1: median {^{S}^{^{qi}}^{(m)}>0}<0 Equation-9

Continuing with the present example, the probability values such as for instance, 4 p-values say {p1, p2, p3, p4} each associated with a model may be obtained based on a non-parametric method used herein, i.e. the Wilcoxon signed rank test. Comparator 335 may sort the p-values to obtain a corresponding model for example, m₀ with the most significant p-value. It will be known to those skilled in the art that the most significant p-value is the one with the lowest value in the set of values and therefore will best fit the model.
p≡pₘ₀=min{p1, p2,p3, p4} Equation-10

Therefore the model with the most significant p-value that best fits may be assigned the genotype and hence the call is made.

Some embodiments of comparator 335 may apply the above described methods to probe sets associated with both the sense and the anti-sense together. An alternative embodiment may include comparator 335 applying the methods to probe sets of each of the strands individually, for example, by separating the sense and the anti-sense strand. By applying the same models and methods to the separate strands, p-values for the forward or sense strand denoted as p_{f}, and the reverse strand, denoted as pᵣ may be obtained. Comparator 335 may then assign a genotype call according to the minimum p-value associated with either the forward or the reverse strand.
Pmin=min{pf,pr} Equation-11

For example, if for a given model p_{f}=0.02 and pᵣ=0.06, the more significant p-values is 0.02 associated with the forward strand. Based on these conditions a genotype call is made.

In alternative cases, the results associated with the sense and the antisense strands may not support the same model. Therefore the user needs to decide if either the strands should support a particular model say AA, or should it be a no call. For example, if p_{f}=A and pᵣ=B, it becomes ambiguous to make a call since it can either be an AA, AB or a BB call. Therefore to reduce the ambiguity, a user may select one particular call to be used in association with the probe sets in question.

Some embodiments of comparator 335 may employ alternative approaches to making genotype calls. For example, a simple sample based dynamic model, may be employed that includes a deviance based-likelihood method where the emission intensities are log normally distributed. In the present example, the deviance based likelihood function may use a parametrical statistical testing method such as a deviance based likelihood function where the first likelihood may be restricted to a small number of parameters, such as for instance, the probe sets or quartets for alleles A and B. The deviance based likelihood can be given for four genotype models, i.e. Null, AA, BB, AB models in a probe set or quartet.

$$\begin{matrix}AA \text{:} {L}_{q} ( AA ) = \begin{matrix}{( log {PM}_{q}^{b} - log {MM}_{q}^{a} )}^{2} + \\ {( log {PM}_{q}^{b} - log {MM}_{q}^{b} )}^{2}\end{matrix} \\ BB \text{:} {L}_{q} ( BB ) = \begin{matrix}{( log {PM}_{q}^{a} - log {MM}_{q}^{a} )}^{2} + \\ {( log {PM}_{q}^{a} - log {MM}_{q}^{b} )}^{2}\end{matrix} \\ AB \text{:} {L}_{q} ( AB ) = \begin{matrix}{( log {PM}_{q}^{a} - log {MM}_{q}^{b} )}^{2} + \\ {( log {PM}_{q}^{a} - log {MM}_{q}^{b} )}^{2}\end{matrix} \\ NULL \text{:} {L}_{q} ( NC ) = \frac{1}{3} [ {L}_{q} ( AA ) + {L}_{q} ( BB ) + {L}_{q} ( AB ) ]\end{matrix} \qquad Equation \text{-} 12$$

where ‘a’ and ‘b’ refer to alleles and ‘q’ indicates the number of restricted parameters, for example, the number of probe sets.

In some embodiments, multiple probe sets may be included. For example, probe array 152 may include a plurality of probe sets which gives a BB genotype call. In order to account for all the probe sets under investigation, a second deviance based likelihood including all the probe sets are represented by Q.

$$\begin{matrix}AA \text{:} L ( AA ) = {\sum }_{q = 1}^{Q} {L}_{q} ( AA ) \\ BB \text{:} L ( BB ) = {\sum }_{q = 1}^{Q} {L}_{q} ( BB ) \\ AB \text{:} L ( AB ) = {\sum }_{q = 1}^{Q} {L}_{q} ( AB ) \\ NULL \text{:} ( NC ) = {\sum }_{q = 1}^{Q} {L}_{q} ( NC )\end{matrix} \qquad Equation \text{-} 13$$

where Q=total number of probe sets

Comparator 335 may apply what may be referred to as transformation methods to the data for the purpose of reduction in the signal to noise ratio. Transformations such as for instance, the Geman-McClure transformation, hereafter referred to as the GM transformation may be used to transform the likelihood equation in order to reduce the number “outliers” in the data. For example, the deviance based likelihood equation derived initially with restricted parameters such as for instance, number of probe sets or quartets, squares (i.e. the squared exponential function such a X²) the outliers which tends to dominate the likelihood. Therefore a transformation such as the GM-transformation, may be a remedy for the effect of outliers on genotype calls, failures of normality, linearity and homoscedasticity i.e., constancy of the variance of a measure over the levels of the factor under study. For example:

$$Equation \text{-} 14 \text{:} GM - Transformation g ( r ) = \frac{{r}^{2}}{{C}^{2} + {r}^{2}}$$

where C is a constant with a default value of 3.5. This default value of C=3.5 may be obtained when r=2 is set as the cutoff point and r is viewed as an outlier if

$$\vert r \vert > \frac{C}{\sqrt{3}} .$$

Comparator 335 may apply the transformation to the initial derivation of the deviance based likelihood to obtain the following equations.

$$AA \text{:} {L}_{q} ( AA ) = {g ( log {PM}_{q}^{b} - log {MM}_{q}^{a} )}^{2} + {g ( log {PM}_{q}^{b} - log {MM}_{q}^{b} )}^{2} \\ BB \text{:} {L}_{q} ( BB ) = {g ( log {PM}_{q}^{a} - log {MM}_{q}^{a} )}^{2} + {g ( log {PM}_{q}^{b} - log {MM}_{q}^{b} )}^{2} \\ AB \text{:} {L}_{q} ( AB ) = {g ( log {PM}_{q}^{a} - log {MM}_{q}^{b} )}^{2} + {g ( log {PM}_{q}^{a} - log {MM}_{q}^{b} )}^{2} \\ NULL \text{:} {L}_{q} ( NC ) = \frac{1}{3} [ {L}_{q} ( AA ) + {L}_{q} ( BB ) + {L}_{q} ( AB ) ] \qquad Equation \text{-} 15$$

where g=3.5 from the GM-transformation.

Correspondingly, the second deviance based likelihood for the all probe calls in the array may incorporate the transformation.

$$AA \text{:} L ( AA ) = {\sum }_{q = 1}^{Q} {L}_{q} ( AA ) \\ BB \text{:} L ( BB ) = {\sum }_{q = 1}^{Q} {L}_{q} ( BB ) \\ AB \text{:} L ( AB ) = {\sum }_{q = 1}^{Q} {L}_{q} ( AB ) \\ NULL \text{:} L ( NC ) = {\sum }_{q = 1}^{Q} {L}_{q} ( NC ) \qquad Equation \text{-} 16$$

where Q=total number of probe sets

In some embodiments comparator 335, may apply alternative statistical models based on some assumptions and parameters about the distribution of the data. Parametric inferential statistical methods are mathematical procedures for statistical hypothesis testing which assume that the distributions of the variables being assessed have certain characteristics. For example, parametric tests such as ANOVA are based on the underlying distributions that are normally distributed and the variances of the distributions being compared are similar. Alternatively the Pearson correlation coefficient assumes normality. Therefore using a parametric approach, a statistical model for example, F₀ may be formulated as

$${F}_{0} = \frac{Q ( {L}_{1} - {L}_{0} )}{{L}_{0}}$$

where
^{L}₀=min{L(AA),L(AB),L(BB),L(NC)} and
^{L}₁=min{L(AA),L(AB),L(BB),L(NC)}\^{L}₀

The value for ^{L}₀ may be calculated by taking the first minimum likelihood value for the calls made. For example, if the calls made are AA=6, AB=7, BB=9, NC=2, then
^{L}₀=2 which is the minimum likelihood that the call made is a no call (NC).

In a second statistical formulation, ^{L}₁ assumes the second minimum number, for example, the second minimum number according to the values given earlier would be 6, which is a call for AA on the site. Therefore ^{L}₁=3 and ^{L}₀=2. i.e. ^{L}₁=6/2, where 6 is the next minimum likelihood value, that the call AA is made.

Alternatively in some embodiments, a different model for example, modified partitioning around mediods, hereafter referred to as the MPAM, may be used to determine the genotypes. The MPAM method may be employed to calculate what may be referred to as a relative allele score hereafter referred to as the RAS value that are obtained for both the sense and the antisense strands. It will be appreciated by those skilled in the relevant art that the RAS value is calculated to demonstrate and visualize the various clustering properties of the SNPs. For example the RAS value associated with a probe set with alleles A and B may include perfect match probes, designated as PM for example, PMA for allele A and PMB for allele B and mismatch probes designated as MM, for example, MMA for allele A and MMB for allele B can be mathematically represented as:

Derivation-1
MM=(MMA+MMB)/2
A=max(PMA−MM, 0)
B=max(PMB−MM, 0)
RAS=A/(A+B)

The conditions required to obtain a defined RAS value is (A+B)>0. Alternatively undefined RAS values may be obtained in conditions such as for instance, if (A+B)=0, or if MM is larger than one of the PMA and PMB to give a value 0 or 1. These values may not be a fair comparison between the signal of allele A and the signal of allele B.

In some embodiments of the invention, two approaches may be used to overcome the problems caused by an undefined and a misleading RAS value. In one embodiment a feature using all the matches and mismatches for example, if the values of PMA−MM or PMB−MM is too small for example, a negative number or a number smaller than a positive number such as for instance C, then a number may be added to these differences to make the smaller difference at least C. Mathematically this can be represented as shown below.

Derivation-2
MM=(MMA+MMB)/2
A′=PMA−MM
B′=PMB−MM
F=max (C−min(A′,B′),0), Where C>0 and C is small
A=A′+F
B=B′+F
RAS2=A/(A+B)

Where RAS2 is the new RAS value which is always defined by a number that is equal to at least C i.e. a smallest positive number. This is because A and B cannot be small. Different values for C can be used for example 5, 10, 20.

For example, if PMA=3500,
MMA=3300
PMB=2999
MMB=2700
MM=(3500+2700)/2=3000
A=500; B=0
RAS1=1

Alternatively another approach to handle this problem is by considering only the perfect match cells for both allele A and B. This reduces the probes by 50% since the mismatches are not considered thus influencing the quality of the clustering. The approach uses a nonlinear transformation to make signals such as for instance, R to move toward 0 and 1 and keep it unchanged at R=0.5. The transformation is therefore symmetric for signals R and (1−R) and can be mathematical represented as shown below.

Derivation-3
R=PMA/(PMA+PMB)
R′=(R−D)/(1−2D), where D is nonnegative and smaller than 0.5
RAS3=max(min (R′,1),0)

Where RAS3 is the modified RAS value which is always defined sine PMA>0 and PMB>0. Various values for D may be used such as for instance D may be equal to 0.05, 0.1, 0.15, and 0.2. For example, R=3500/(3500+2999)=0.5385443
RAS3=R′=(0.5385443−0.1)/(1−2*0.1)=0.5481804

The result shows that the value supports an AB call.

Data Reliability Tester 345: In some embodiments, the sequence data manager 323, may forward the genotype call results to data reliability tester 345 in order to test the reliability of genotype calls obtained from the comparator 335. For example, the quality of the statistics may be calculated so that the significance level and the accuracy of the calls made by the dynamic model with non-parametric test hereafter referred to as the DMNP may be determined. This may help to control the quality of the genotype calls made and give more reliable calls. Therefore a quality statistics may be set as,
medianS=median{S^{q1}(m₀),S^{q2}(m₀), . . . ,S^{qi}(m₀)} Or meanS=mean{S^{q1}(m₀)S^{q2}(m₀), . . . ,S^{qi}(m₀)} Equation-17
while the most significant p-value as the best fit model that makes a call may be shown as
p<α Equation-18

α could be set to obtain the confidence interval for example, α=0.05 for a confidence level of 95%.

In some embodiments, it will be understood by those skilled in the relevant art that the α-value holds a significant role in the call confidence to determine the statistical models that would be best suited to make the call. For example, if the α=0.1, then the DMNP model may be selected. Alternatively if the α=0.025, a different model for example the dynamic model with parametric test (DMP) may be selected to make the call.

Continuing with the example of the dynamic model used for the genotype calls, the data manager 323, may forward the genotype call results to the data reliability tester 345 in order to check the quality of the statistics built. For instance to check the significance level, the accuracy of the calls made and determine the reliability of the call based on parametric statistical inferences, an F-test can be used to determine a cut-off point wherein it is decided if the calls are to be made or not. For example, an F-test can be given by;
F₀=F(1,Q−1)
where ^{F}₀ is the statistical model defined earlier and ^{F(1,Q-1)} is the F-statistic that may be given by

$$\frac{( {ESS}_{R} - {ESS}_{UR} ) / q}{{ESS}_{UR} / ( n - k )} \approx F ( q , n - k )$$

where ESS is the error of the sum of squares, R is the restricted parameters and UR is the unrestricted parameters, q indicates the number of restricted parameters and k is the number of parameters.

The cut off value for the probability of the F-statistic can be given as i.e. the p-value may be given by ^{p=P{F>F}^{₀}^{}} for example, based on the condition ^{p<α}^{₀}, the decision is made to make a call or a no call. For example, ^{α}^{₀} can be set to be the cut-off value for the p-value which gives the confidence interval. It will be appreciated by those skilled in the relevant are that the terms α and ^{α}^{₀} refer to the same cut off value that determines the confidence level of the call in the dynamic model. In this context, α refers to the confidence level for the DMNP model while ^{α}^{₀} refers to the confidence level for the DMP model.

It will be known to those skilled in the relevant art that, the genotyping algorithms may be designed to work with one or more samples such as, for instance, multiple samples over multiple experiments one or more genotyping algorithms may be applied. For example, the Hardy-Weinberg equilibrium rule may be applied for each target sequence or SNP across all samples. It will be known to those skilled in the related art that the Hardy-Weinberg principle provides a baseline to determine whether or not gene frequencies have changed in a population and thus whether evolution has occurred. The Hardy-Weinberg equilibrium applies the chi-square test to compare the observed and the expected genotype frequency distribution between the three genotypes, for example, AA, AB and BB. The chi-square test gives us the probability that the difference between observed and the expected frequencies are due to chance. Therefore the Hardy-Weinberg equilibrium when applied each SNP across all samples, may eliminate low quality calls or at least provide warnings to the user.

Data Optimizer 370: In some embodiments, algorithms may be employed to asses the quality of each probe sets ability to provide reliable data. For example, it may be possible to identify probe sets that do not provide high quality data and reduce the number of probe sets (i.e. remove the low quality probe sets) used without significant loss of accuracy of the genotype call rate.

For example, data optimizer 370 may perform what may be referred to as a probe reduction process that determines the probe sets that optimally identify each target sequence or SNP in a sample thus reducing the number of probes needed. In the present example, reducing the number of probes needed to identify a target sequence or SNP may result in a reduction of space on probe array 152 required to identify each SNP. In the present example, the number of probe sets necessary to provide reliable information may be reduced from 14 to 10, that could result in at least 28-30% more space on the array to tile probes for additional SNPs. Such probe reduction techniques may include the identification of the single probe pair for each target sequence that best approximates the average difference for all the base pairs.

Sample Optimization: One embodiment of probe reduction may include what may be referred to as Dynamic Modeling methods that, for instance, may be used to optimize the reliability of the probe sets to interrogate a set of particular target sequences or SNP's in a single sample. For example, the reliability may be optimized by fixing the sample with a number that may be referred to as PQ and is associated with the number of probe sets used to interrogate the sample. Optimizer 370 may implement a dynamic modeling method, such as the method described above with respect to comparator 335 for making genotype calls, using a p-value cut off at 1 in order to generate as many genotype calls as possible. The output assigns a value for each probe set based, at least in part, on the assumption that the genotype call is correct. In the present example, the output corresponding to a particular target sequence or SNP may include 14 values associated with probe sets interrogating both the sense and the anti-sense strands. Optimizer 370 ranks the p-values associated with each probe set such that the more significant p-value has a lower rank.

Target Sequence or SNP Optimization: Also, in the same or other embodiment, each probe set that interrogates a target sequence or SNP may be optimized by summing the calculated ranks for each probe set from all N samples for each combination of probe sets that interrogate the target sequence or SNP resulting in an aggregated rank value. Optimizer 370 then ranks each of the different combinations based for each SNP, at least in part, upon the aggregated rank value, where the combination with the minimum rank is the optimal combination of probe sets for the SNP.

In addition or alternatively, for each probe set assigned as a no call, optimizer 370 assigns the associated likelihood value of negative infinity, which penalizes each probe set that does not have a model that fits well other than a No Call assignment. Optimizer 370 then formulates a vector with N likelihood differences from all the N samples for the probe set. Next, optimizer 370 applies Wilcoxon's signed rank test to the formulated vector that results in a p-value. Optimizer 370 ranks the p-values for each probe set associated with a SNP that may, for instance, include 14 probe sets such that the combination of the probe sets with the most significant p-values may be the optimal combination for the SNP.

Aggregated Optimization: In some implementations, optimizer 370 may perform an aggregated optimization method for all SNPs with all N samples by first performing the sample optimization methods and second by performing the sample optimization methods.

Sequence data manager 323 may then assemble and store the results from data filters 325, analysis models comparator 335, data reliability tester 345, and/or data optimizer 370 into one or more genotype call data files 350. Data 350 may include genotype calls corresponding to all samples, or alternatively there may be a separate data file 350 that corresponds to each sample. For example, the genotype call results from sample emission intensity data files 145′, 145″, and 145′″ may be combined into one sample genotype data file 350. In the present example, that could be a separate sample genotype data file 350 for each sample emission intensity data files 145.

Output Manager 360: Output manager 360 may receive the one or more data files 350 from manager 323.

In some embodiments the output manager 360 may arrange the genotype calls from each sample and pass them to input-output controllers 130. Controllers 130 then correspond with the display devices 180 to present the user with the genotype results in a graphical user interface, hereafter referred to as a GUI.

Many visualization tools are available that present the user with the results of the analysis. However a user friendly visualization tool aids the user to easily understand the results, the overall quality of the results and provides the user a level of flexibility to decide the parameters, for example, selectable cut off values in order to obtain the desired genotype results. This tool may further aid in the linkage study or the association when applied to the whole genome and may give an in depth coverage of the whole genome being studied.

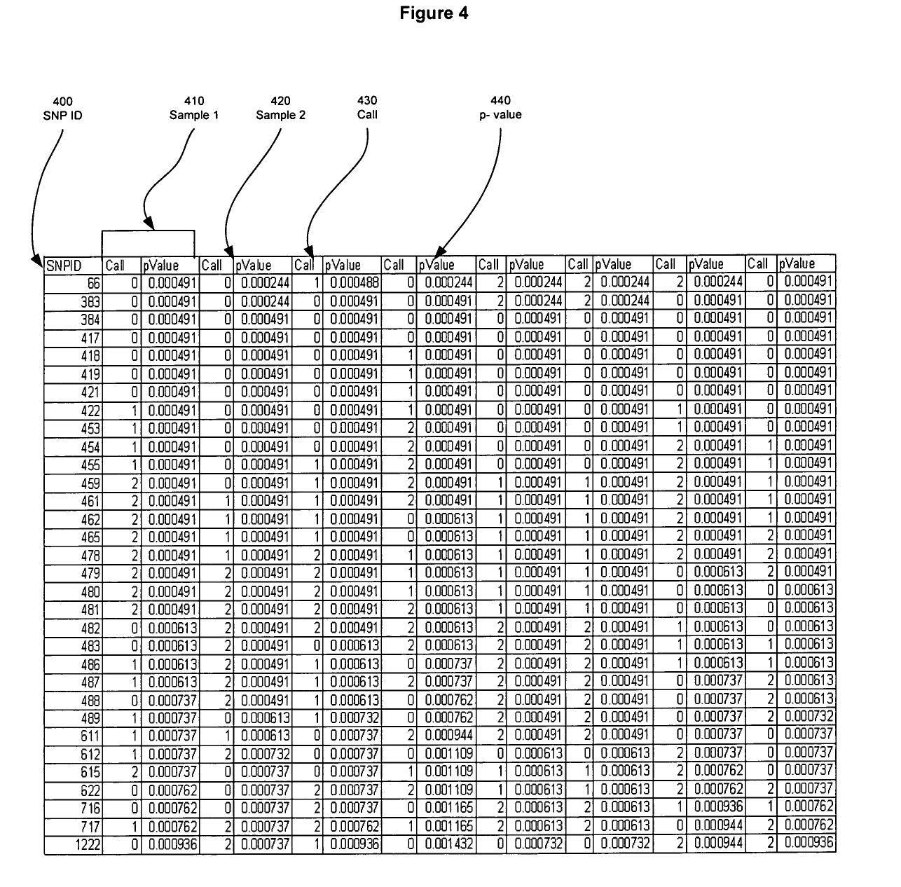

FIG. 4 is a snapshot of a graphical representation of a GUI. The GUI displays a table of the genotype calls and associated p-values associated with a SNP for multiple samples. The rows depict the SNPs represented with distinctive ID's, SNP ID 400 while the columns depict the samples. For the purpose of illustration a total of 11 samples were used in this experiment (all 11 samples not shown in the figure). For example the samples are depicted as sample-1 410, sample-2 420. Each sample comprises calls 430 and p-values 440 for each associated SNP. It will be appreciated by those skilled in the relevant art that the sample size may differ, for example there may be less than 11 samples or more than 11 samples. The calls may be represented by numbers. For example, 0 may depict an AA call, 1 may represent an AB call while 2 may represent a BB call and −1 may be used to represent a no call.

In some embodiments the GUI may also present color map 530 to provide a visual representation of the genotype calls. The color maps may use the basic red, blue and green to depict the various calls. For example, the color red may depict a homozygous AA call while blue may depict a homozygous BB call. Accordingly green may depict a heterozygous AB call. Alternatively a black or a white color on the color map may depict a no call. Therefore the color maps in the illustration presented show the genotype calls for the multiple SNPs across multiple samples, which in this case is 114 SNPs across 11 samples. It will be appreciated by those skilled in the relevant art that the colors used to depict the genotype need not necessarily be only those mentioned above and that any other alternative colors available on a color palette may be used. It will also be appreciated by those skilled in the relevant art that the number of SNPs and the sample size used here is only for the purpose of illustration and that the number could be a number lower or higher than that mentioned herein.

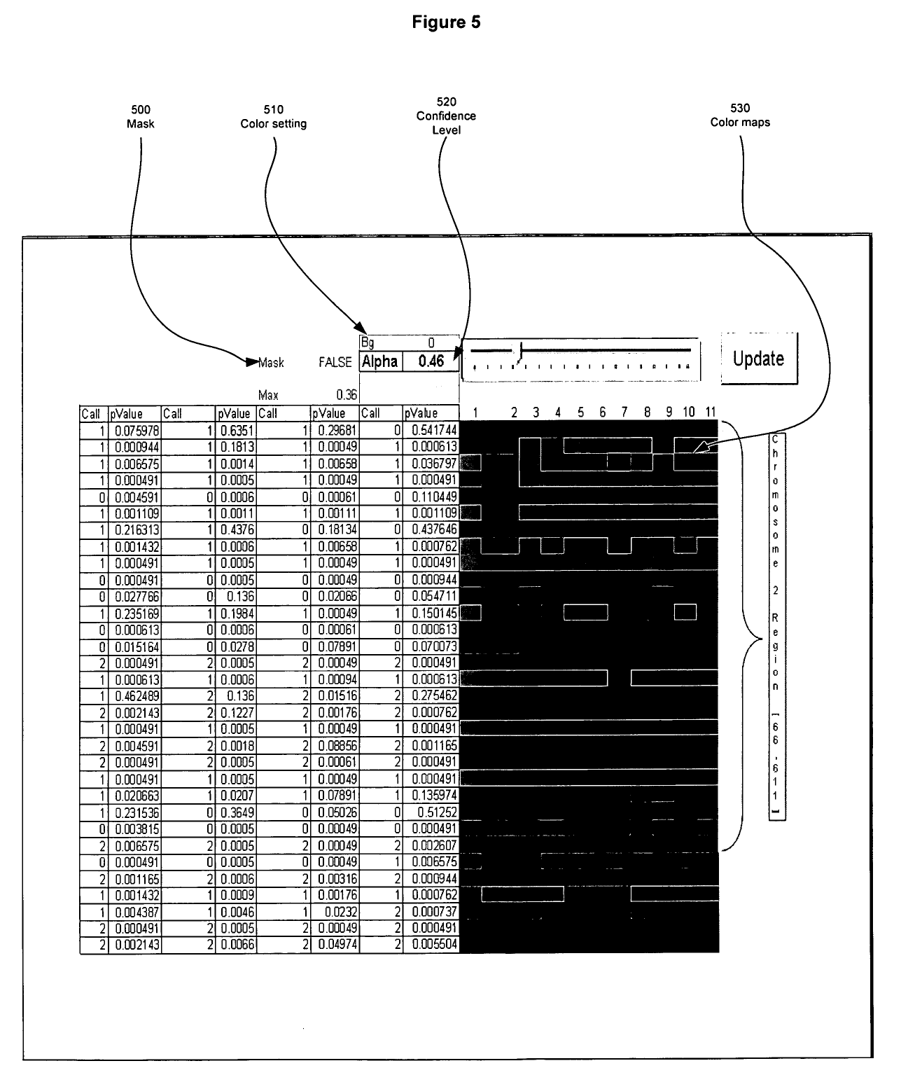

In some embodiments the GUI may present user interactive options with the help of which the user may change one or more parameters such as, for instance, a threshold value that may vary the genotyping call results. For example, the user interface may have an option to change the color settings of the color maps such as for instance, in FIG. 5; color setting 510 may be changed from 0 to a 299 (not shown in figure). These numbers signify the color settings, for example, by setting color setting 510 to a 0, the user may obtain the color map genotype call results in the black extreme of the color palette. Alternatively changing the color setting 510 to a 299 may enable the user to visualize the color maps in the other extreme of the color palette wherein the number 299 signifies the color white. It will be appreciated by those skilled in the relevant art that the number may not be strictly adhered to 0 and 299 and can be any number between these two extremes. After the user decides the color scale to be used for identification, the user may set the other options to obtain the results.

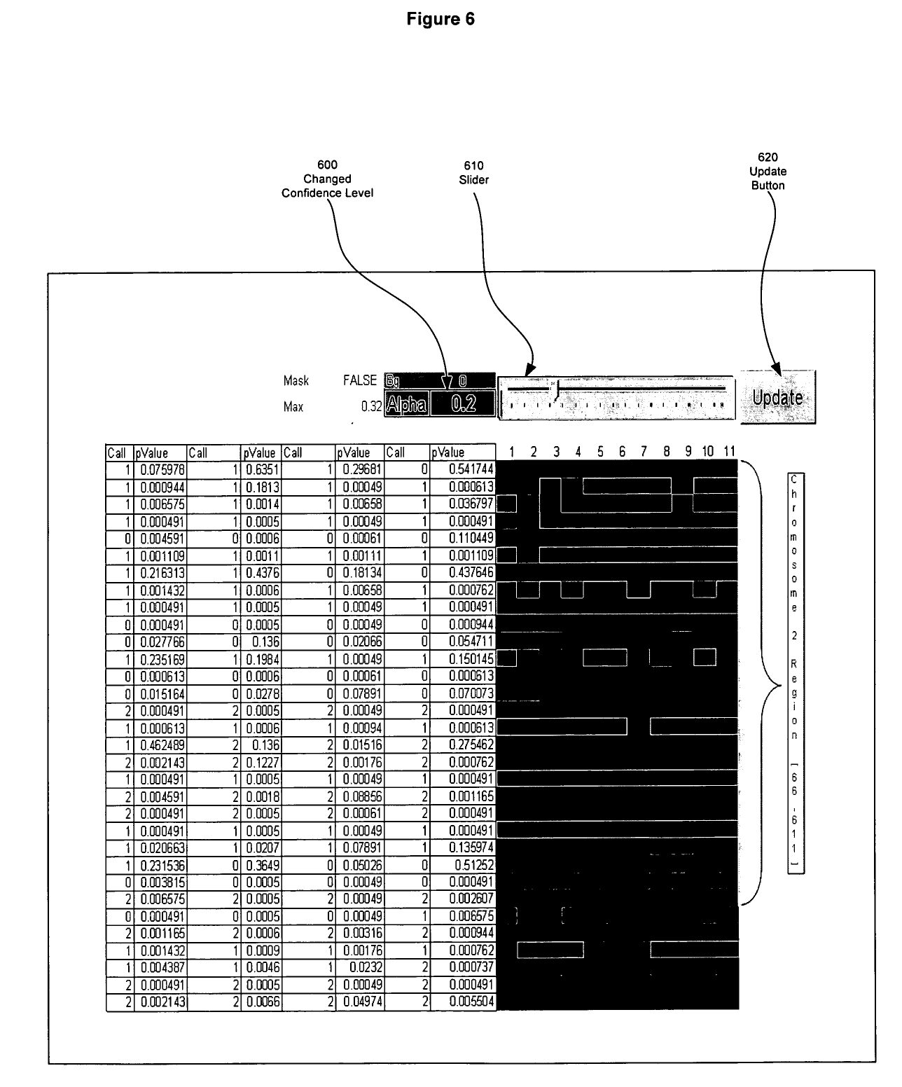

For example, some implementations may include a user selectable alpha value. Confidence level 520 shows the cut off or threshold value that may affect how a genotyping call is made. Illustrated in FIG. 6, slider 610 allows the user to change the confidence value. In other words the sliders provide a means for user selecting values that may be aid in the interpretation of quality of genotype calls. The cutoff value may be an arbitrary or an empirical number. As used in this context, an arbitrary number refers to a number based on or subject to the judgment or preference of the user while empirical number refers to a number relying on or derived from observation or experiment i.e. a number that is decided earlier on by the user. Such change in the cutoff values based on the user judgment enables the system to present to the user a visual representation of the changes in the genotype call. For example by adjusting the slider, the cells associated with the calls above the cutoff or below the cutoff may change to, for example, black or white which depicts a no call in that region for the genotype.

Alternatively, the user may type in the desired cutoff value in the box next to the ‘alpha’ and then chose the update 620 button. On choosing the update 620 button the system updates the genotype calls of the multiple SNPs across the multiple samples and presents the user with the updated results based on the new specification of the user. The system therefore allows the user to visualize the changes and further decide if the user wants to change the cutoff or accept the results based on the cutoff specified.

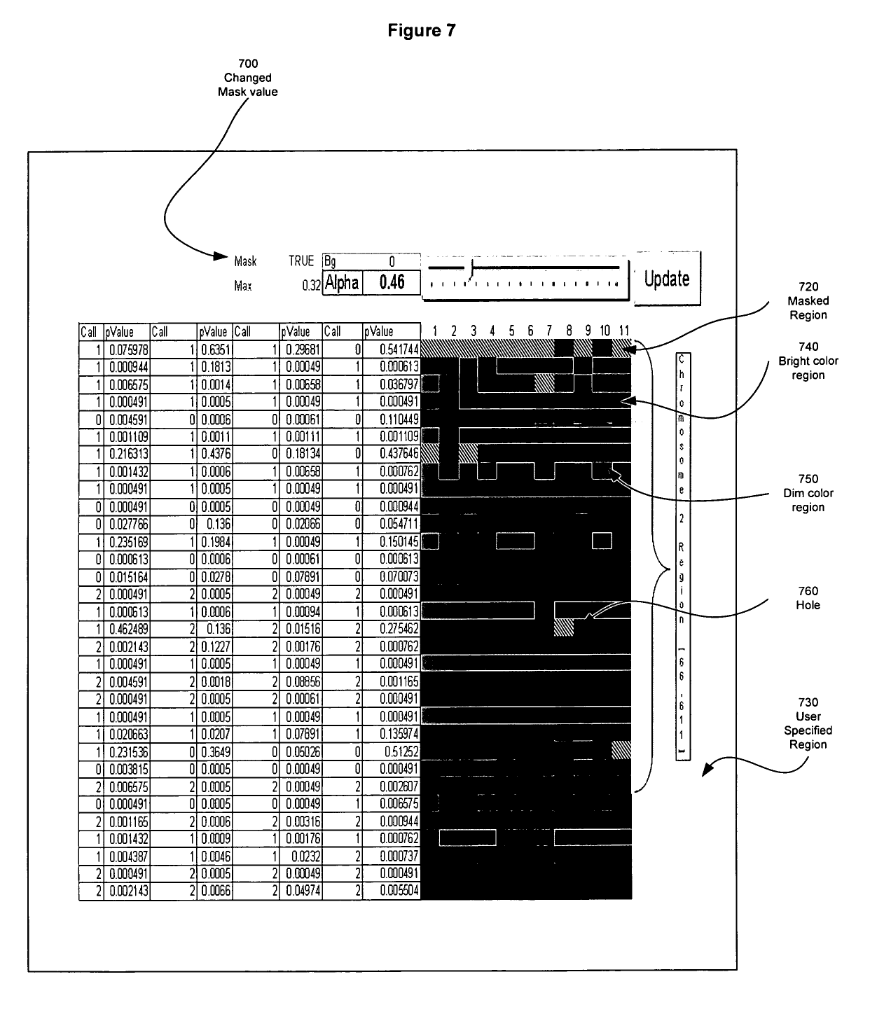

In some embodiments the adjustable confidence level may change the brightness of the color displayed by the genotypes in the color maps. The brightness may be adjusted with the call confidence level which as mentioned earlier may be user specified. For example, if the specified confidence level is highly significant then the genotype which matches the threshold value will exhibit a brighter color when compared to a genotype call with a low confidence level. In FIG. 7, the bright region 740 represents the region which exhibits a high confidence level while the dim region 750 represents the region with a low confidence level for that genotype. The color proportions may be adjusted by inputting the color coding into the confidence matrix in a way that the higher, the cell matches with the confidence level, the more brighter will the cell be and alternatively, the lower it matches with the chosen confidence level, the less brighter or dimmer will the cell be thus helping in the visual interpretation about the confidence of that particular genotype call at the user specified confidence level.

In some implementations other options include an option to ‘mask’ 700 some regions. Mask as used in this context refers to the process of shading a region which matches the user specified criteria in order to enable easy visual identification. For example, if the user wants to check the low confidence calls and know exactly where the no calls lie, then the user may change the mask values for example from FALSE (as seen in FIGS. 5 and 6) to TRUE (as seen in FIG. 7). The FALSE option which may be present in the user interface by default is depicted on the color map as black regions and refer to the No call regions of the genotype calls. If the user opts to place a mask on the color map, the user can change the FALSE to TRUE thereby instructing that a mask is to be placed. In such cases, the no call regions are masked and are depicted by shaded regions. However in certain implementations, after applying the mask, few black regions may be seen. For example, as seen in FIG. 7, a black region 760 is seen after the mask is applied. This region depicts a hole or in simple terms a gap. For example, these holes 760 may signify a gap in the linkage association studies of the genome. A gap as used herein refers to a sequence position or range on a strand of DNA or RNA where a nucleotide or a segment of nucleotides are missing. Therefore this makes it easy for the user to visually interpret the no call regions in the genotype and decipher the various regions that may be a hole or a gap.

In some implementations, the number of the SNPs and the sample sizes may be numerous and may not be of complete interest to the user. For example, in the genotype analysis of a whole genome, the user may be interested only in a specific region say for instance, the chromosome 2 region which is illustrated as the user specified region 730 in FIG. 7. By choosing one specific region of interest in the whole genome, the system allows the user to concentrate on the results obtained in the region of interest and in this way the user is allowed to visually analyze the results in a sub-region to sub-region manner.

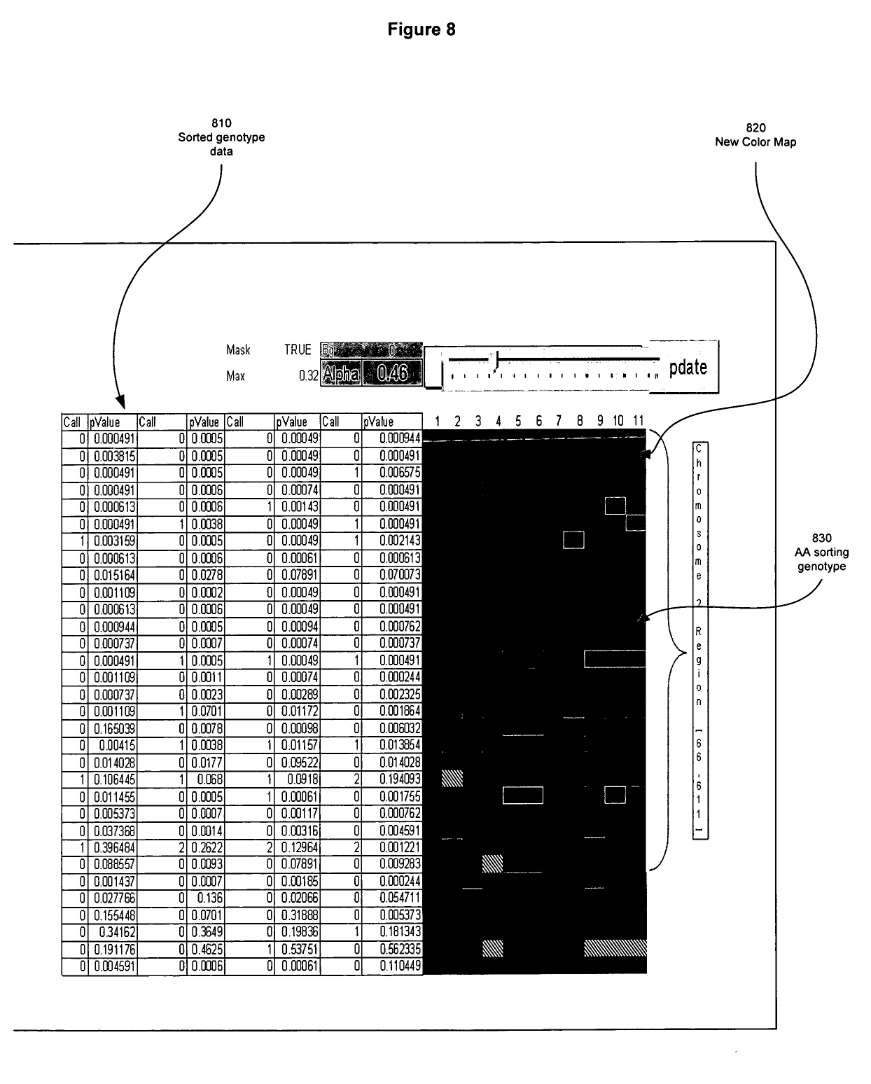

Data Sorting: In some embodiments, the user may be allowed to sort the genotype data in order to obtain color maps with different effects to enhance the visual inference of the genotyping. This in effect would provide the user with various visual representations based on the different parameters specified by the user. For example, the user may want to sort the genotype calls 810, based on the call types i.e. 0, 1 or 2 that correspond to AA, AB or BB calls. Therefore the user can use a program that may perform the sort function. The data sorting program may be implemented in any programming language such as for instance C, C++, Java, or Perl. The sorted data may be obtained in a separate file or directly applied to the simulation to obtain the color maps. In this manner the user is allowed to order the genotypes obtained in the way the user desires. For example, the user may want to obtain a color map that may be sorted in a way that the AA may be sorted to the upper region of the color map, the AB may be sorted to appear in the middle of the color map while all the BB genotypes may be sorted to appear in at the end of the color map. FIG. 8 illustrates the sorting of the genotype data by the user 175 wherein the user chose to sort the data by the criteria mentioned above. As seen in the figure, the AA genotype calls have been sorted at the upper level of the color map while the AB genotype calls are sorted at the middle of the color map (not shown in the figure) and finally the BB genotype calls are sorted towards the end of the color map (not shown in the figure).

The previous example is used for the purposes of illustration only and should not be limiting in any way. A variety of colors or other graphical representations may be used to indicate a variety of possible features.

Having described various embodiments and implementations, it should be apparent to those skilled in the relevant art that the foregoing is illustrative only and not limiting, having been presented by way of example only. Many other schemes for distributing functions among the various functional elements of the illustrated embodiment are possible. The functions of any element may be carried out in various ways in alternative embodiments. For example, some or all of the functions described as being carried out by output manager 360 could be carried out by sequence data manager 323, or these functions could otherwise be distributed among other functional elements. Also, the functions of several elements may, in alternative embodiments, be carried out by fewer, or a single, element. For example, the functions of output manager 360 and sequence data manager 323 could be carried out by a single element in other implementations. Similarly, in some embodiments, any functional element may perform fewer, or different, operations than those described with respect to the illustrated embodiment. Also, functional elements shown as distinct for purposes of illustration may be incorporated within other functional elements in a particular implementation. For example, the functions performed by the two servers could be performed by a single server or other computing platform, distributed over more than two computer platforms, or other otherwise distributed in accordance with various known computing techniques.

Also, the sequencing of functions or portions of functions generally may be altered. Certain functional elements, files, data structures, and so on, may be described in the illustrated embodiments as located in system memory of a particular computer. In other embodiments, however, they may be located on, or distributed across, computer systems or other platforms that are co-located and/or remote from each other. For example, any one or more of data files or data structures described as co-located on and “local” to a server or other computer may be located in a computer system or systems remote from the server. In addition, it will be understood by those skilled in the relevant art that control and data flows between and among functional elements and various data structures may vary in many ways from the control and data flows described above or in documents incorporated by reference herein. More particularly, intermediary functional elements may direct control or data flows, and the functions of various elements may be combined, divided, or otherwise rearranged to allow parallel processing or for other reasons. Also, intermediate data structures or files may be used and various described data structures or files may be combined or otherwise arranged. Numerous other embodiments, and modifications thereof, are contemplated as falling within the scope of the present invention as defined by appended claims and equivalents thereto.

1) A method for calling the genotype of a sample, comprising;
receiving emission data for one or more target sequences each hybridized to a plurality of probe sets, wherein each of the plurality of probe sets comprises a plurality of probe features;
calculating a set of values for each of the plurality of probe sets associated with each target sequence;
selecting one of the set of values for each of the probe sets associated with each target sequence, wherein the value is selected if it is greater than a reference value;
determining a significance value from the selected values of all the probe sets associated with each target sequence; and
producing a genotype call for each target sequence based upon the significance value.

2) The method of claim 1, wherein:
the emission data includes data from detected fluorescent emissions.

3) The method of claim 1, wherein:
the one or more target sequences include DNA sequences.

4) The method of claim 3, wherein:
the DNA sequences include single nucleotide polymorphism sequences.

5) The method of claim 1, wherein:
each of the plurality of probe sets is disposed on a probe array.

6) The method of claim 1, wherein:
each of the plurality of values includes a log likelihood value.

7) The method of claim 1, wherein:
each of the set of values is calculated based upon one or more assumptions.

8) The method of claim 7, wherein:
each of the one or more assumptions comprises a genotype assumption.

9) The method of claim 8, wherein:
the genotype assumption is selected from the group consisting of a null assumption, a homozygous assumption, and a heterozygous assumption.

10) The method of claim 1, wherein:
the selected value corresponds to a preliminary genotype call for the probe set.

11) The method of claim 1, wherein:
the significance value is statistically determined.

12) The method of claim 11, wherein:
the statistical determination includes a non-parametric method.

13) The method of claim 1, wherein:
the genotype call is selected from the group consisting of AA, BB, AB, and null.

14) The method of claim 1, further comprising:
storing the genotype call for each target sequence.

15) The method of claim 1, further comprising:
displaying the genotype call for each target sequence.

16) A computer for calling the genotype of a sample comprising system memory with executable code stored thereon, wherein the executable code is enabled to perform a method, comprising;
receiving emission data for one or more target sequences each hybridized to a plurality of probe sets, wherein each of the plurality of probe sets comprises a plurality of probe features;
calculating a set of values for each of the plurality of probe sets associated with each target sequence;
selecting one of the set of values for each of the probe sets associated with each target sequence, wherein the value is selected if it is greater than a reference value;
determining a significance value from the selected values of all the probe sets associated with each target sequence; and
producing a genotype call for each target sequence based upon the significance value.

17) The computer of claim 16, wherein:
the emission data includes data from detected fluorescent emissions.

18) The computer of claim 16, wherein:
the one or more target sequences include DNA sequences.

19) The computer of claim 18, wherein:
the DNA sequences include single nucleotide polymorphism sequences.

20) The computer of claim 16, wherein:
each of the plurality of probe sets is disposed on a probe array.

21) The computer of claim 16, wherein:
each of the plurality of values includes a log likelihood value.

22) The computer of claim 16, wherein:
each of the set of values is calculated based upon one or more assumptions.

23) The computer of claim 22, wherein:
each of the one or more assumptions comprises a genotype assumption.

24) The computer of claim 23, wherein:
the genotype assumption is selected from the group consisting of a null assumption, a homozygous assumption, and a heterozygous assumption.

25) The computer of claim 16, wherein:
the selected value corresponds to a preliminary genotype call for the probe set.

26) The computer of claim 16, wherein:
the significance value is statistically determined.

27) The computer of claim 26, wherein:
the statistical determination includes a non-parametric method.

28) The computer of claim 16, wherein:
the genotype call is selected from the group consisting of AA, BB, AB, and null.

29) The computer of claim 16, the method further comprising:
storing the genotype call for each target sequence.

30) The computer of claim 16, the method further comprising:
displaying the genotype call for each target sequence.

31) A method for calling the genotype of a sample, comprising;
generating emission data for one or more target sequences each hybridized to a plurality of probe sets, wherein each of the plurality of probe sets comprises a plurality of probe features;
receiving the emission data;
calculating a plurality of values for each of the plurality of probe sets associated with each of the one or more target sequences;
selecting one of the set of values for each of the probe sets associated with each target sequence, wherein the value is selected if it is greater than a reference value;
determining a significance value from the selected values of the probe sets associated with each target sequence; and
producing a genotype call for each target sequence based upon the significance value.

32) A system for calling the genotype of a sample, comprising;
a scanner that generates emission data for one or more target sequences each hybridized to a plurality of probe sets, wherein each of the plurality of probe sets comprises a plurality of probe features; and
a computer comprising system memory with executable code stored thereon, wherein the executable code is enabled to perform a method, comprising;
receiving the emission data;
calculating a plurality of values for each of the plurality of probe sets associated with each of the one or more target sequences;
selecting one of the set of values for each of the probe sets associated with each target sequence, wherein the value is selected if it is greater than a reference value;
determining a significance value from the selected values of the probe sets associated with each target sequence; and
producing a genotype call for each target sequence based upon the significance value.
